(19) United States
(12) Patent Application Publication
(10) Pub. No.: US 2025/0129033 A1
(43) Pub. Date: Apr. 24, 2025

(54) METAP2 INHIBITORS AND USES THEREOF

(71) Applicant: Yissum Research Development Company of the Hebrew University of Jerusalem Ltd., Jerusalem (IL)

(72) Inventors: Ofra BENNY, Jerusalem (IL); Arie DAGAN, Jerusalem (IL)

(73) Assignee: Yissum Research Development Company of the Hebrew University of Jerusalem Ltd., Jerusalem (IL)

(21) Appl. No.: 18/833,008

(22) PCT Filed: Feb. 15, 2023
(86) PCT No.: PCT/IL2023/050157

§ 371 Date: Jul. 25, 2024

Related U.S. Application Data
(60) Provisional application No. 63/310,730, filed on Feb. 16, 2022.

(51) Int. Cl. C07D 303/40; A61K 31/336; A61P 35/00; C07D 303/46

(52) U.S. Cl. CPC C07D 303/40; A61K 31/336; A61P 35/00; C07D 303/46

(57) ABSTRACT

Novel inhibitors of MetAp2 enzymatic activity and uses thereof are provided.

### TECHNOLOGICAL FIELD

The invention generally relates to Novel MetAp2 inhibitors and uses thereof.

### BACKGROUND

Anti-angiogenic drugs have the ability to prevent, inhibit, and regress newly formed blood vessels. Several forms of angiogenesis inhibitors exist, ranging from endogenous proteins and small molecule cytokine antagonists, to antibodies and tyrosine kinase inhibitors [1-4]. Historically, the main target of anti-angiogenic drugs in clinical development has been to block the vascular endothelial growth factor (VEGF) pathways. However, other angiogenic factors such as basic fibroblast growth factor (bFGF), platelet-derived growth factor (PDGF) and inflammatory cytokines also play critical roles in neovascularization.

One of the most effective compounds which demonstrated angiostatic activity in preclinical studies is derived from fumagillin, a natural product secreted by fungi with anti-infective properties. Synthetic analogues of fumagillin and its biologically active metabolite, fumagillol, were found to be powerful anti-angiogenic compounds capable of preventing growth of new blood capillaries. After screening more than one hundred compounds, the most potent anti-angiogenic analogue of fumagillin was found to be chloro acetylcarbamoylfumagillol (TNP-470) [5].

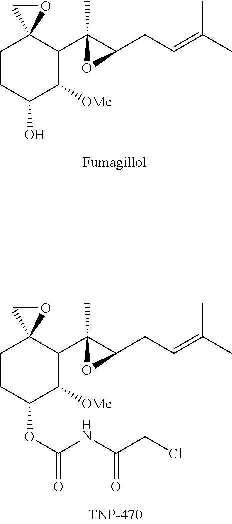

The molecular target of TNP-470 is methionine aminopeptidase-2 (MetAP2), an enzyme responsible for the removal of methionine from newly synthesized proteins. MetAp2 is overexpressed in proliferating endothelial cells, and therefore inhibition of MetAp2 is assumed to lead to selective inhibition of angiogenesis [6-10].

The activity of TNP-470 has been demonstrated in mice, rats, rabbits, hamsters, dogs, monkeys, and humans. It has also been investigated in eight clinical studies involving more than three hundred patients [11-19]. In these studies, TNP-470 was administered as an intravenous formulation at different dosing levels and schedules. Overall, activity was observed in more than twelve tumor types. Despite its encouraging efficacy, the fumagillol analogue TNP-470 has a short half-life in plasma (minutes) [17, 19] leading to poor pharmacokinetics. Additionally, in high doses it was found to cause adverse side-effects such as reversible neurotoxicity in some patients [11].

### BACKGROUND

1. Folkman, J., Angiogenesis and angiogenesis inhibition: an overview. Exs, 1997. 79: p. 1-8.
2. Folkman, J., Fighting cancer by attacking its blood supply. Scientific American, 1996. 275(3): p. 150-4.
3. Folkman, J., Tumor angiogenesis and tissue factor [comment]. Nature Medicine, 1996. 2(2): p. 167-8.
4. Folkman, J., Angiogenesis inhibitors generated by tumors. Molecular Medicine, 1995. 1(2): p. 120-2.
5. Ingber, D., et al., Synthetic analogue of fumagillin that inhibit angiogenesis and suppress tumour growth. Nature, 1990. 348: p. 555-557.
6. Chun, E., et al., Novel inhibitors targeted to methionine aminopeptidase 2 (MetAP2) strongly inhibit the growth of cancers in xenografted nude model. Int J Cancer, 2005. 114(1): p. 124-30.
7. Griffith, E. C., et al., Molecular recognition of angiogenesis inhibitors fumagillin and ovalicin by methionine aminopeptidase 2. Proc Natl Acad Sci USA, 1998. 95(26): p. 15183-8.
8. Turk, B. E., et al., Selective inhibition of amino-terminal methionine processing by TNP-470 and ovalicin in endothelial cells. Chem Biol, 1999. 6(11): p. 823-33.
9. Wang, J., et al., Tumor suppression by a rationally designed reversible inhibitor of methionine aminopeptidase-2. Cancer Res, 2003. 63(22): p. 7861-9.
10. Zhang, Y., et al., Cell cycle inhibition by the anti-angiogenic agent TNP-470 is mediated by p53 and p21WAF1/CIP1. Proc Natl Acad Sci USA, 2000. 97(12): p. 6427-32.
11. Bhargava, P., et al., A phase I and pharmacokinetic study of TNP-470 administered weekly to patients with advanced cancer Clin Cancer Res, 1999. 5: p. 1989-1995.
12. Herbst, R., et al., Safety and pharmacokinetic effects of TNP-470, an angiogenesis inhibitor, combined with paclitaxel in patients with solid tumors: evidence for activity in non-small-cell lung cancer. J Clin Oncol, 2002. 20: p. 4440-4447.
13. Kruger, E., Figg, W D., TNP-470: an angiogenesis inhibitor in clinical developmentfor cancer. Expert Opin Investig Drugs, 2000. 9: p. 1383-1396.
14. Kudelka, A., et al., A phase I study of TNP-470 administered to patients with advanced squamous cell cancer of the cervix. Clin Cancer Res 1997. 3: p. 1501-1505.
15. Kudelka, A., C. Verschraegen, and E. Loyer, Complete remission of metastatic cervical cancer with the angiogenesis inhibitor TNP-470. N Engl J Med, 1998. 338: p. 991-992
16. Logothetis, C., et al., Phase I trial of the angiogenesis inhibitor TNP-470 for progressive androgen-independent prostate cancer. Clin Cancer Res, 2001. 7: p. 1198-1203.
17. Moore, J. D., et al., Phase I dose escalation pharmacokinetics of O-(chloroacetylcarbamoyl) fumagillol (TNP-470) and its metabolites in AIDS patients with Kaposi's sarcoma. Cancer Chemother Pharmacol, 2000. 46(3): p. 173-9.
18. Stadler, W., Kuzel, T., Shapiro, C., Sosman, J., Clark, J., Vogelzang, NJ., Multi-institutional study of the angiogenesis inhibitor TNP-470 in metastatic renal carcinoma. J Clin Oncol, 1999. 17: p. 2541-2545.
19. Tran, H., et al., Clinical and pharmacokinetic study of TNP-470, an angiogenesis inhibitor, in combination with paclitaxel and carboplatin in patients with solid tumors. Cancer Chemother Pharmacol, 2004. 54: p. 308-314.
20. U.S. Pat. No. 5,204,345.

### General Description

Angiogenesis inhibition was established as an important modality for tumor suppression and spread when combined with chemotherapeutic drugs. While there is a range of inhibitors that reached clinical approval, many of them were not sufficiently efficient or were found to carry various side effects. Therefore, finding new angiogenic inhibitors, with high potency and drug-like properties, has been a target to establishing new venues in cancer treatment, especially given the lower toxicity profile of these agents compared with chemotherapies.

Many studies have established that MetAP2 plays an important role in the development of various types of cancer and that the specific downregulation of human MetAP2 expression by an antisense oligonucleotide had a predominant effect on endothelial cell proliferation. In the context of the development of the novel agents disclosed herein, the inventors have realized the involvement of MetAP2 in lymphangiogenesis, indicating a dual action of MetAp2 in both vascular and lymphatic capillary formation. Therefore, there was a rationale for positioning MetAp2 as a useful target for the treatment of primary cancers, and generally metastatic diseases.

As noted above, one of the most effective known inhibitors of MetAp2 originates from the natural compound Fumagillin. This molecule was isolated from Aspergillus fumigatus Fresenius, and the synthetic analogue, O-chloroacetylcarbamoyl fumagillol or TNP-470 (also known as AGM-1470), was one of the most potent analogs of fumagillin as demonstrated in angiogenesis cell models and one of the first anti-angiogenic small molecule drugs to undergo clinical trials. However, the development of this derivative was hindered by major clinical drawbacks related to dose-depending side effects.

A large series of fumagillin derivatives has been synthesized and their biological activity was tested. While the majority of these compounds has been determined inactive or not of sufficient safety, a unique group of compounds disclosed herein were determined safe and highly active in suppressing endothelial cell proliferation and in arresting MeAp2 enzymatic activity.

More specifically it was established that compounds of the invention markedly suppressed the proteolytic activity of MeAp2. The inhibition of MetAp2 enzymatic activity led to angiogenesis blockage and suppressed tumor growth. Furthermore, it was found that the presence of compounds of the invention in human melanoma cell line as well as in primary endothelial cells impaired cell proliferation. The in vitro observations correlated with in vivo results, which demonstrated substantial anti-tumor activity in different tumor-bearing mice models. These results support the broad biological effect and superiority of compounds of the invention.

Histological analyses showed that endothelial cells re-modulation was affected. Immunofluorescence of extracted murine tumors tissue sections showed that in treated tissues blood vessels positive cells were organized more sporadically and less collectively as vessels, compared with untreated groups. Moreover, compounds of the invention reduced the cellular proliferation in treated tumors.

Given the promising in vivo results, compounds of the invention have been formulated for treatment or prevention of a variety of conditions, in a variety of delivery forms, including delivery in nanoparticle-based encapsulation. The capacity to enhance bioavailability of lipophilic drugs, as well as improving their retention and stability in vivo, using 10- to 100-fold less of a dose than the free drug was established. The cellular availability in monolayer cultures and in 3D multicellular cultures was also developed. It was found that the relatively high concentrations that were required for suppressing cell proliferation in vitro was attributed to the limited transport into cells. The inhibition effect of the encapsulated compounds on the proliferation of endothelial and A375 cells showed a significant improvement of bioavailability of with almost 20-fold reduction in dose compared with the free compounds. Unlike 2D cell cultures, in the 3D culture, cells assemble and interact spatially thus showing more physiologically relevance and provide better prediction of drug efficacy compared to monolayer cell cultures.

Taken together, data presented herein demonstrate the superiority and promising therapeutic properties of compounds of the invention in prevention and treatment of cancer progression. Compounds of the invention demonstrated inhibitory activity and generally effective inhibition in blood vascularization and tumor progression in tumor-bearing mice. These significant results were mainly attributed to the compounds anti-angiogenic and anti-cancer activity.

The invention thus provides novel compounds, compositions, uses and method of treatment and prophylaxis.

In a first aspect, there is provided a compound of the general Formula (I):

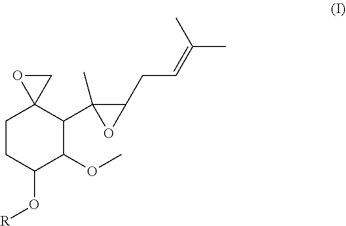

wherein
R is a carbocyclyl comprising a ring structure of two or more rings.

A compound of Formula (I) contains at least 6 chiral carbon centers. Each of these chiral centers may be of either the (R) or (S) configuration. Thus, compounds provided herein may be in enantiomerically pure forms, or may be provided in stereoisomeric or diastereomeric mixtures.

The invention thus provides an enantiomer of a compound of Formula (I).

The invention further provides a chiral compound of Formula (I).

The invention further provides a compound of Formula (I) as a MetAp2 inhibitor and further as an anti-angiogenic agent.

Compounds of the invention are further purposed as MetAp2 inhibitors in methods of inhibiting MetAp2 activity or in methods of preventing or treating angiogenesis, as further disclosed herein.

The invention further provides use of a compound disclosed herein in the preparation of compositions, e.g., pharmaceutical compositions. The compositions may be used experimentally or therapeutically in connection with treatment or prevention of angiogenesis, or any other condition or disease disclosed herein.

Compounds of the invention are compounds of Formula (I), wherein the “carbocyclyl” functionality (variant R in a compound of Formula (I)) is any non-linear alkylene structure that comprises only single bonds, or which comprises one or more double bonds or triple bonds, or an aromatic ring. The carbocycle is a multi-cyclic ring system comprising 2 or more or at least two rings or ring systems. The carbocyclyl may be composed of two or more rings, wherein at least one of the rings is a 6-membered ring structure, which may be joined to another ring or ring structure (being a 6-membered ring or a structure of a different size ring) in a fused, bridged or spiro-fashion.

The carbocyclyl may be a hydrocarbon composed of only carbon atoms or may be in a form of a heterocarbocyclyl further containing at least one heteroatom selected from the group consisting of O, N, and S. The carbocyclyl may include one or more double bonds or may be fully aromatic.

Typically, the carbocycle comprises a 6-membered ring structure that is fused, bridged or spiro-connected to another ring or ring structure to form a further 6-membered ring. For example, the carbocyclyl may be a group that is a fusion of two or three 5-membered or 6-membered rings. Alternatively, the carbocyclyl may be a bridged 5-membered or 6-membered ring. Non-limiting examples of carbocyclyl groups include adamantly and derivatives thereof, norbornanyl and derivatives thereof, norbornanyl and derivatives thereof, norbornenyl and derivatives thereof, steroidyl and derivatives thereof, camphoryl and camphor derivatives, camphenyl and camphene derivatives, tricyclo(2.2.1.0(2,6))heptanyl and derivatives thereof, tetracyclo [3.2.0.0(2,7).0(4,6)]heptanyl and derivatives thereof, and others.

Adamantyl is derived from adamantane of the structure

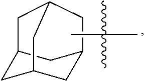

wherein the adamantyl may have any substitution and may be associated to a compound of Formula (I) through any of the adamantly carbon atoms (designated by a dashed line).

Norbornanyl is derived from norbornane of the structure

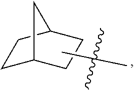

wherein the derivative may have any substitution and may be associated to a compound of Formula (I) through any of the norbornanyl carbon atoms (designated by a dashed line).

Norbornenyl is derived from norbornane of the structure

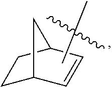

wherein the derivative may have any substitution and may be associated to a compound of Formula (I) through any of the norbornenyl carbon atoms (designated by a dashed line). The steroidyl may be any steroid known in the art. Camphoryl is derived from camphor of the structure

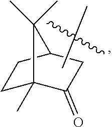

wherein the derivative may have any substitution and may be associated to a compound of Formula (I) through any of the camphoryl carbon atoms (designated by a dashed line). Camphenyl is derived from camphene of the structure

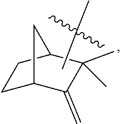

wherein the derivative may have any substitution and may be associated to a compound of Formula (I) through any of the camphenyl carbon atoms (designated by a dashed line).

Tricyclo(2.2.1.0(2,6))heptanyl is derived from tricyclo(2.2.1.0(2,6))heptane of the structure

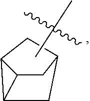

wherein the derivative may have any substitution and may be associated to a compound of Formula (I) through any of the tricyclo(2.2.1.0(2,6))heptanyl carbon atoms (designated by a dashed line).

Tetracyclo[3.2.0.0(2,7).0(4,6)]heptanyl is derived from tetracyclo[3.2.0.0(2,7).0 (4,6)]heptane of the structure

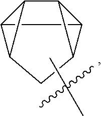

wherein the derivative may have any substitution and may be associated to a compound of Formula (I) through any of the tetracyclo[3.2.0.0(2,7).0(4,6)]heptanyl carbon atoms (designated by a dashed line).

The association of the carbocycle to the oxygen atom may be direct or via a spacer or a linker moiety as further disclosed herein.

In some embodiments, the carbocyclyl is or comprises an aromatic ring or aromatic ring structure or a heteroaromatic ring or ring structure. The aromatic ring structure may comprise between 6 and 10 carbon atoms which may or may not be fused to another aromatic or non-aromatic carbocycle. The heteroaromatic ring may comprise between 5 and 10 carbon atoms and 1 or more (1, 2, or 3) heteroatoms selected from N, O and S.

In some embodiments, the carbocyclyl is not an aromatic ring or ring system.

Each of the carbocyclyl groups may be connected to the oxygen atom of a compound having a structure of Formula (I) via any carbon atom of the carbocyclyl group. For example, where the carbocyclyl is adamantyl, the adamantyl may be substituted to the oxygen atom of a compound of Formula (I) via carbon 1 or 2 of the adamantyl ring structure. Where norbornanyl and derivatives thereof are concerned, the norbornanyl or derivative may be linked through carbons 1, 2 or 7 of the ring structure.

Each carbocyclyl group may be substituted by one or more groups or atoms, on any carbon atom of the ring structure, providing a variety of carbocyclyl derivatives with modified (improved) toxicities and activities. Generally speaking, derivatives of any of the recited carbocyclyl groups may be alkyl derivatives, hydroxylated derivatives, aromatic derivatives, halogenated derivatives, acid derivatives (comprising —COOH or other acidic groups), amine derivatives, sulfonated derivatives and others. Non-limiting examples of substitutions may include —C₁-C₃alkyl, —C₂-C₄alkenyl, —C₁-C₃alkylhalide (wherein the alkyl is substituted by one or more halogen atoms, including I, Br, Cl or F), hydroxyl, carboxyl, carboxylate, —C₆-C₁₀aryl (such as phenyl, napthyl), hydroxy-C₁-C₃alkyl, sulfate, sulfonate, sulfonamide, sulfonic acid and one or more halides. Where a carbon atom is said not to be substituted, the particular carbon atom maintains the original bonds and substitutions. The substitution may be by a single substituting group, by two or more substituting groups, by multiple substituting groups or the carbocyclyl group may be fully substituted.

In some embodiments, variant R may be bonded to the oxygen atom of a compound of Formula (I) directly or via a spacer or linker moiety X as depicted in a compound of Formula (IA):

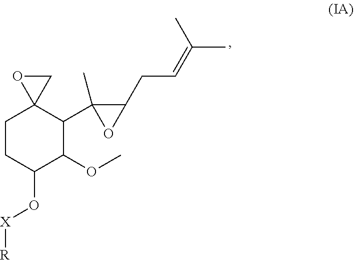

wherein R is as defined herein and wherein X is a functional group linking carbocyclyl R with the oxygen atom of a compound of Formula (I). Group X may be an atom or a group of atoms. In some embodiments, X comprises at least two atoms, e.g., —C═O—.

In some embodiments, X is absent and R is directly bonded to the oxygen atom.

In some embodiments, X is selected from —C₁-C₅alkylene (e.g., methylene, ethylene, propylene, etc), which may or may not be substituted; —C₂-C₅alkenylene (comprising one or more double bonds), which may or may not be substituted; —C₆-C₁₀arylene (such as phenylene or naphthylene), which may or may not be substituted; —C₅-C₁₀heteroarylene, which may or may not be substituted, and which comprises one or more heteroatoms selected from N, O and S; —C₁-C₅alkylene-C₆-C₁₀arylene, which may or may not be substituted; —C₁-C₅alkylene-C₅-C₁₀heteroarylene, which may or may not be substituted, and which comprises one or more heteroatoms selected from N, O and S; —C(═O)—C₆-C₁₀arylene, which may or may not be substituted; —C(═O)—C₅-C₁₀heteroarylene, which may or may not be substituted, and which comprises one or more heteroatoms selected from N, O and S; —C(═O)—C₆-C₁₀arylene-NH—C(═S)—NH—, which may or may not be substituted; —C(═O)—C₅-C₁₀heteroarylene-NH—C(═S)—NH—, which may or may not be substituted, and which comprises one or more heteroatoms selected from N, O and S; —NH—C(═S)—NH—; —C₁-C₅alkylene-NH—C(═S)—NH—; —NH—C(═S)—NH—C₁-C₅alkylene; —C(═O)—; —O(C═O)—; —C₁-C₅alkylene-C(═O)—; —C₁-C₅alkylene-O(C═O)—; —NH—C(═O)—; —NH—C(═O)—C₁-C₅alkylene-C(═O)—; —SO₂NH—; —NH—SO₂— and others.

As used herein, “alkylene” refers to a straight, branched or cyclic, in certain embodiments straight or branched, divalent aliphatic hydrocarbon group, having from 1 to 5 carbon atoms (inclusive), or 1, 2, 3, 4, or 5 carbon atoms. Alkylene groups include, but are not limited to, methylene (—CH₂), ethylene (—CH₂CH₂—), propylene (—(CH₂)₃—), etc. The propylene may be linear propylene or iso-propylene. The butylene may be linear butylene, secondary butylene, tertiary butylene. The pentylene may be linear pentylene, tertiary pentylene, isopentylene, secondary pentylene, etc.

The “arylene” refers to a monocyclic or polycyclic divalent aromatic group, having from 6 to 10 carbon atoms and at least one aromatic ring. The arylene may include, but is not limited to, 1,2-, 1,3- and 1,4-phenylene.

As used herein, “heteroarylene” refers to a divalent monocyclic or multicyclic aromatic ring system, having 5 to 10 carbon atoms in the ring(s), and one or more, in some embodiments 1 to 3, heteroatoms selected from nitrogen, oxygen and sulfur.

Where a group comprises a combination of functionalities, such as “—C₁-C₅alkylene-C₆-C₁₀arylene”, each of the alkylene and arylene is defined as herein.

In some embodiments, X is or comprises a —C₁-C₅alkylene group.

In some embodiments, X is or comprises —C₁-C₅alkylene, —C(═O)— and/or —O(C═O)—.

In some embodiments, X is or comprises —C₁-C₅alkylene, wherein the alkylene may be methylene, ethylene, propylene, butylene or pentylene, which may or may not be substituted by a substituent R₄, as defined herein. In some embodiments, the alkylene is methylene or ethylene. In some embodiments, the alkylene is substituted by a halide atom such as F, Cl, Br or I.

In some embodiments, X is —C(═O)—.

In some embodiments, a compound of structure (I) or (IA) is a compound of structure (IB):

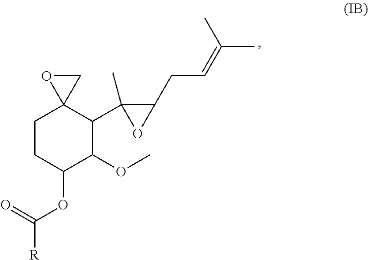

wherein R is as defined herein.

In some embodiments, in a compound of Formula (IA), X is —(C═O)—O—.

In some embodiments, the compound of Formula (I) or (IA) is a compound designated compound (IC):

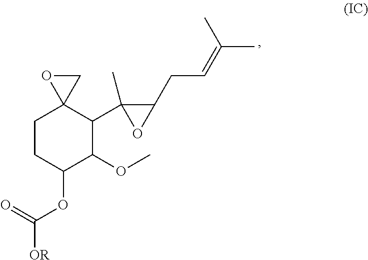

wherein R is as defined herein.

In some embodiments, in a compound of Formula (IA), X is or comprises —C(═O)—C₁-C₅alkylene-NH—C(═S)—NH—.

In some embodiments, a compound of Formula (I) or (IA) is a compound of Formula (ID):

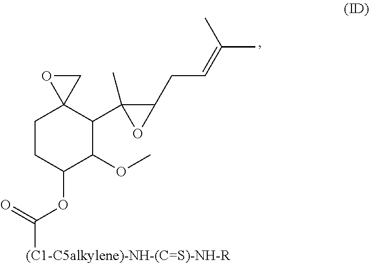

wherein R is as defined herein.

In some embodiments, the —C₁-C₅alkylene may be methylene, ethylene, or pentylene, which may or may not be substituted by a substituent R₄, as defined herein.

In some embodiments, in a compound of Formula (IA), X is or comprises —C(═O)—C₁-C₅alkylene-O(C═O)—.

In some embodiments, a compound of Formula (I) or (IA) is a compound of Formula (IE):

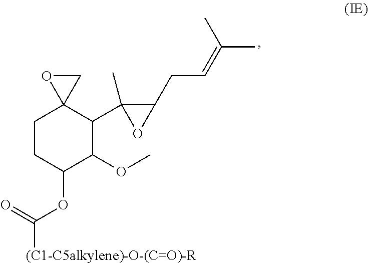

wherein R is as defined herein.

In some embodiments, in a compound of Formula (IA), X is or comprises —C(═O)—NH—C(═O)—.

In some embodiments, a compound of Formula (I) or (IA) is a compound of Formula (IF):

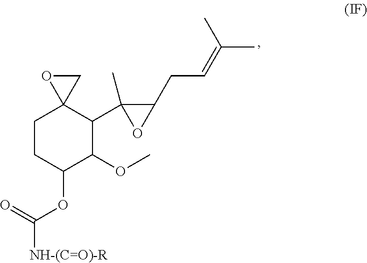

wherein R is as defined herein.

In some embodiments, in a compound of Formula (IA), X is or comprises —NH—C(═O)—C₁-C₅alkylene-C(═O)—.

In some embodiments, a compound of Formula (I) or (IA) is a compound of Formula (IG):

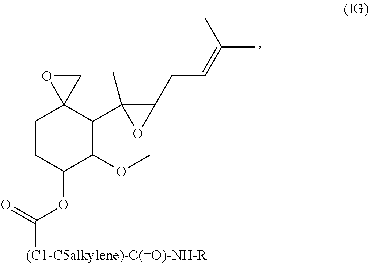

wherein R is as defined herein.

In some embodiments, the —C₁-C₅alkylene may be ethylene or propylene, which may or may not be substituted by a substituent R₄, as defined herein.

In some embodiments, a compound of Formula (I) or (IA) is a compound of Formula (IH):

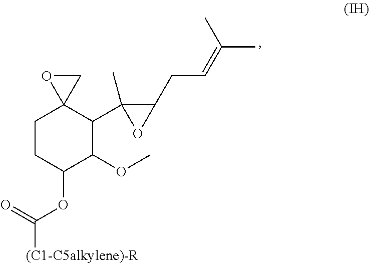

wherein R is as defined herein.

In some embodiments, the —C₁-C₅alkylene may be methylene, ethylene, propylene, butylene or pentylene, which may or may not be substituted by a substituent R₄, as defined herein.

In some embodiments, the —C₁-C₅alkylene may be methylene, ethylene, propylene, butylene or pentylene, which may or may not be substituted by a halogen atom, as defined.

In some embodiments, in a compound of Formula (IA), X is absent.

In some embodiments, in each of the Formulae disclosed herein, the —C₁-C₅alkylene may be selected from methylene, ethylene, propylene, butylene and pentylene.

In some embodiments, in each of the Formulae disclosed herein, the —C₁-C₅alkylene may or may not be substituted, e.g., by a halide atom.

In some embodiments, the —C₁-C₅alkylene is methylene or ethylene or propylene or butylene or pentylene. In some embodiments, the —C₁-C₅alkylene comprises one carbon atom, two carbon atoms, three carbon atoms, four carbon atoms or five carbon atoms.

In some embodiments, the carbocyclyl R is selected from norbornanyl, norbornenyl and adamantyl.

In some embodiments, the carbocyclyl R is adamantyl that is optionally substituted. In some embodiments, the adamantyl may be linked to the oxygen atom of a compound of Formula (I) via carbon 1 or 2 of the adamantly. In some embodiments, the adamantly is of structure (A1) and (A2):

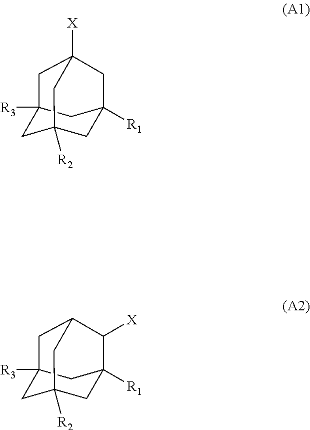

wherein for each of structure (A1) and (A2), independently:
X is a functional group or an atom associating the adamantly group with the oxygen atom of the compound of Formula (I), as disclosed herein; and
wherein each of R₁, R₂ and R₃, independently of the other, may be —H, or may be selected from halide (F, Cl, Br, I), —C₁-C₃alkyl, —C₂-C₄alkenyl, —C₁-C₃alkylhalide, hydroxyl, carboxyl, carboxylate, —C₆-C₁₀aryl, hydroxyalkyl, sulfate, sulfonate, sulfonamide, and sulfonic acid.

In some embodiments, the —C₁-C₅alkyl is methyl or ethyl or propyl or butyl or pentyl. In some embodiments, the —C₁-C₅alkyl is methyl.

In some embodiments, X is absent and the adamantly group is directly bonded to the oxygen atom of the structure of Formula (I), as shown above.

In some embodiments, X is —C(═O)—; —O(C═O)—; —C₁-C₅alkylene-C(═O)—; —C₁-C₅alkylene-O(C═O)—; —NH—C(═O)—; or —NH—C(═O)—C₁-C₅alkylene-C(═O)—, wherein X may or may not be substituted by a variant R₄.

In some embodiments, the adamantly is of structure (B):

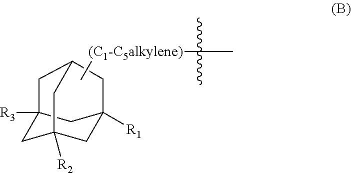

wherein
each of R₁, R₂ and R₃, independently of the other, may be H, or may be selected from —C₁-C₃alkyl, —C₂-C₄alkenyl, —C₁-C₃-alkylhalide, hydroxyl, carboxyl, carboxylate, —C₆-C₁₀aryl, hydroxyalkyl and halide; and

[symbol] indicates a point of connectivity to the oxygen atom of a structure of Formula (I).

In some embodiments, one or more of R₁, R₂ and R₃ is H or a —C₁-C₅alkyl, selected from methyl, ethyl, propyl, butyl and pentyl. In some embodiments, one or more of R₁, R₂ and R₃ is methyl, ethyl or propyl.

In some embodiments, one or more of R₁, R₂ and R₃ is methyl or a halide atom.

In some embodiments, R₄ is absent.

In some embodiments, the adamantly is of structure (C):

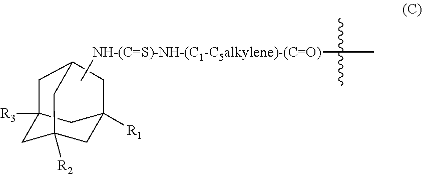

wherein
each of R₁, R₂ and R₃, independently of the other, may be H, or may be selected from —C₁-C₃alkyl, —C₂-C₄alkenyl, —C₁-C₃-alkylhalide, hydroxyl, carboxyl, carboxylate, —C₆-C₁₀aryl, hydroxyalkyl and halide; and
[symbol] indicates a point of connectivity to the oxygen atom of a structure of Formula (I).

In some embodiments, one or more of R₁, R₂ and R₃ is H or a —C₁-C₅alkyl, selected from methyl, ethyl, propyl, butyl and pentyl. In some embodiments, one or more of R₁, R₂ and R₃ is methyl, ethyl or propyl.

In some embodiments, one or more of R₁, R₂ and R₃ is methyl or a halide atom.

In some embodiments, R₄ is absent.

In some embodiments, the adamantly is of structure (D):

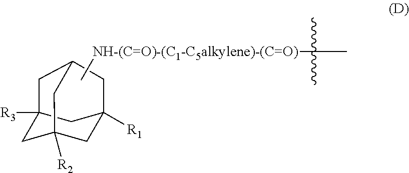

wherein
each of R₁, R₂ and R₃, independently of the other, may be H, or may be selected from —C₁-C₃alkyl, —C₂-C₄alkenyl, —C₁-C₃-alkylhalide, hydroxyl, carboxyl, carboxylate, —C₆-C₁₀aryl, hydroxyalkyl and halide; and
[symbol] indicates a point of connectivity to the oxygen atom of a structure of Formula (I).

In some embodiments, one or more of R₁, R₂ and R₃ is H or a —C₁-C₅alkyl, selected from methyl, ethyl, propyl, butyl and pentyl. In some embodiments, one or more of R₁, R₂ and R₃ is methyl, ethyl or propyl.

In some embodiments, one or more of R₁, R₂ and R₃ is methyl or a halide atom.

In some embodiments, R₄ is absent.

In some embodiments, in each of the Formulae herein, the adamantly is bonded via carbon 1 or 2, as depicted in structure (A1) or (A2) above.

In some embodiments, a compound of Formula (I) is selected from compounds herein designated:
AD-3302;
AD-3281;
AD-3306;
AD-3283;
AD-3305; and
AD-3301.

In some embodiments, a compound of Formula (I) is selected from compounds herein designated:
AD-3295;
AD-3294;
AD-3286;
AD-3287; and
AD-3290.

In some embodiments, in a compound of Formula (I), R is norbornenyl or norbornanyl. In some embodiments, the carbocyclyl is of structure (E):

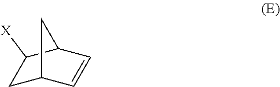

wherein X is as defined herein.

In some embodiments, the carbocyclyl is of structure (F):

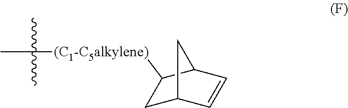

wherein the —C₁-C₅alkylene is selected from methylene, ethylene, propylene, butylene and pentylene. In some embodiments, the alkylene is methylene, ethylene or propylene.

In some embodiments, the carbocyclyl is of structure (G):

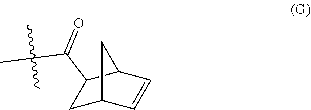 [SMILES: *C(=O)C1CC2C=CC1C2]

wherein the dashed line designates the bond of connectivity to the oxygen atom of a compound of Formula (I).

In some embodiments, X is absent and the norbornenyl or norbornanyl is directly bonded to the oxygen atom of a compound of Formula (I).

In some embodiments, a compound of Formula (G) is herein designated AD-3303.

In some embodiments, a compound of the invention is a compound herein designated:
AD-3280; and
AD-3199.

Excluded from compounds of Formula (I) are compounds in which R is a linear or branched alkyl, that is not a carbocyclyl. Also excluded are straight-chained or branched alkyl groups, alkenyl groups, alkynyl groups. Further excluded are optionally substituted acyl groups such as acyl groups derived from carboxylic acid or its amide groups (e.g. alkanoyl group, aroyl group, aromatic heterocyclic carbonyl group, carbamoyl group, alkoxy carbonyl, phenoxy carbonyl group, etc.) or acyl groups derived from sulfonic acid or its amido groups, (e.g. benzenesulfonic group, sulfamoyl group, etc.). Also excluded are optionally substituted alkyl groups shown such as —C₁-C₂₀ straight-chain or branched alkyl groups optionally having 1-3 substituents. These excluded groups may be alkyl groups that are epoxidated at optional positions; such as methyl, ethyl, benzyl, etc. Specifically excluded are optionally substituted alkyl groups including substitutions such as amino, lower alkyl amino (e.g. methylamino, ethylamino, isopropylamino, etc.), di-lower alkyl amino (e.g. dimethylamino, diethylamino, etc.), nitro, halogen (e.g. fluorine, chlorine, bromine, iodine, etc.), hydroxyl, lower alkoxy (e.g. methoxy, ethoxy, etc.), cyano, carbamoyl, carboxyl, lower alkoxycarbonyl (e.g. methoxycarbonyl, ethoxycarbonyl, etc.), carboxy lower alkoxy (carboxymethoxy, 2-carboxyethoxy, etc.), optionally substituted phenyl, aromatic heterocyclic groups (5-6 membered aromatic heterocyclic groups containing 1-4 hetero-atoms such as nitrogen, oxygen, sulfur, etc., such as 2-furyl, 2-thienyl, 4-thiazolyl, 4-imidazolyl, 4-pyridyl, etc.).

Further excluded are optionally substituted alkanoyl groups.

The invention further provides a compound being any one of:

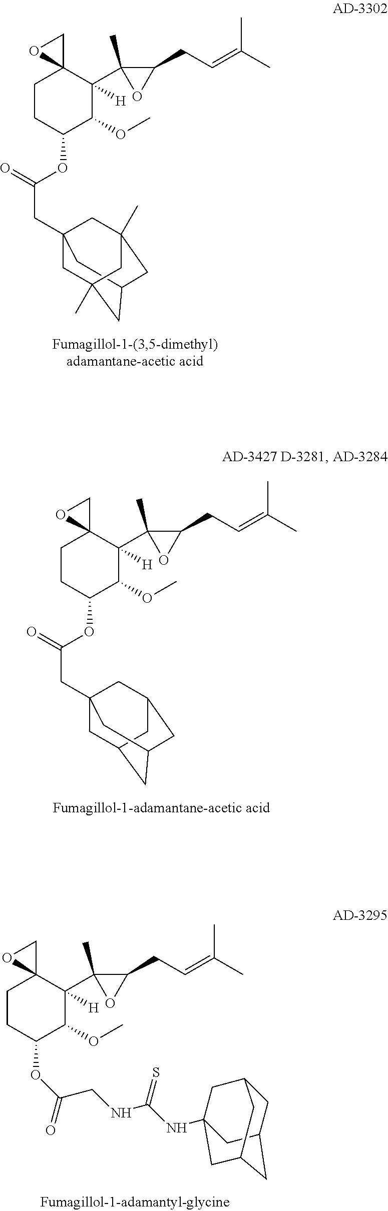 [SMILES: CO[C@@H]1[C@H](OC(=O)CC23CC4CC(C)(CC(C)(C4)C2)C3)CC[C@]2(CO2)[C@H]1[C@@]1(C)O[C@@H]1CC=C(C)C ; CO[C@@H]1[C@H](OC(=O)CC23CC4CC(CC(C4)C2)C3)CC[C@]2(CO2)[C@H]1[C@@]1(C)O[C@@H]1CC=C(C)C ; CO[C@@H]1[C@H](OC(=O)CNC(=S)NC23CC4CC(CC(C4)C2)C3)CC[C@]2(CO2)[C@H]1[C@@]1(C)O[C@@H]1CC=C(C)C]

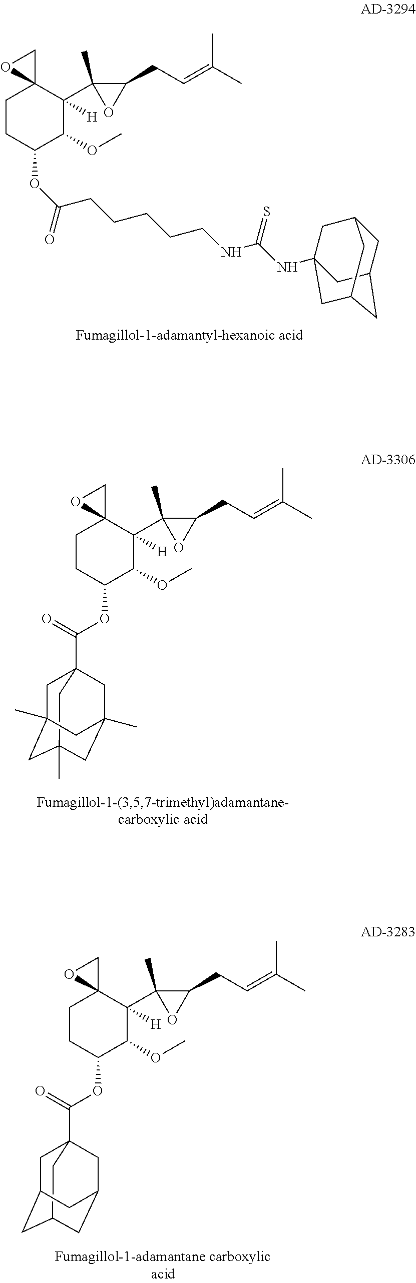 [SMILES: CO[C@@H]1[C@H](OC(=O)CCCCCNC(=S)NC23CC4CC(CC(C4)C2)C3)CC[C@]2(CO2)[C@H]1[C@@]1(C)O[C@@H]1CC=C(C)C ; CO[C@@H]1[C@H](OC(=O)C23CC4(C)CC(C)(CC(C)(C4)C2)C3)CC[C@]2(CO2)[C@H]1[C@@]1(C)O[C@@H]1CC=C(C)C ; CO[C@@H]1[C@H](OC(=O)C23CC4CC(CC(C4)C2)C3)CC[C@]2(CO2)[C@H]1[C@@]1(C)O[C@@H]1CC=C(C)C]

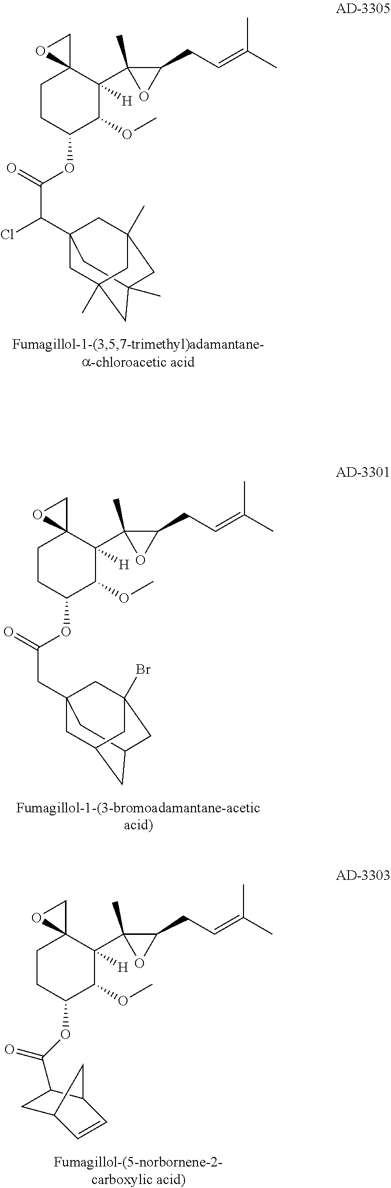 [SMILES: CO[C@@H]1[C@H](OC(=O)C(Cl)C23CC4(C)CC(C)(CC(C)(C4)C2)C3)CC[C@]2(CO2)[C@H]1[C@@]1(C)O[C@@H]1CC=C(C)C ; CO[C@@H]1[C@H](OC(=O)CC23CC4CC(CC(Br)(C4)C2)C3)CC[C@]2(CO2)[C@H]1[C@@]1(C)O[C@@H]1CC=C(C)C ; CO[C@@H]1[C@H](OC(=O)C2CC3C=CC2C3)CC[C@]2(CO2)[C@H]1[C@@]1(C)O[C@@H]1CC=C(C)C]

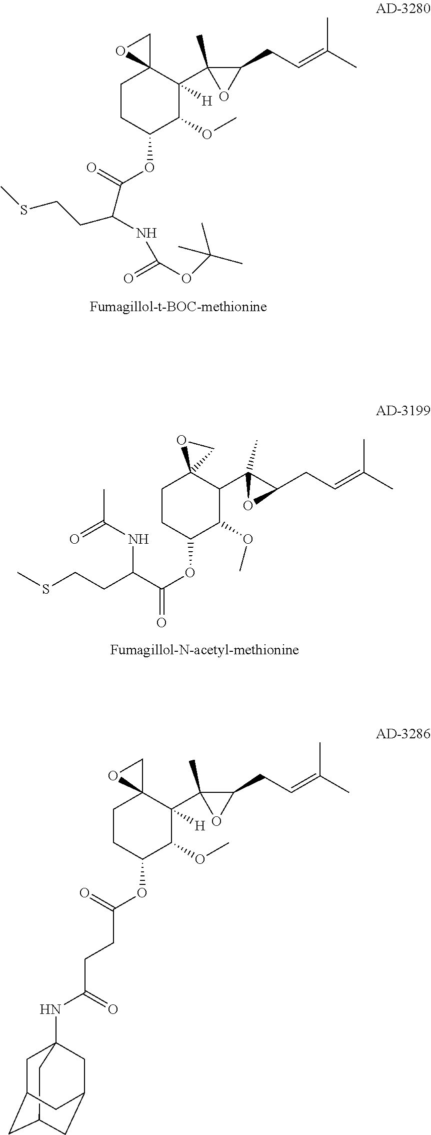

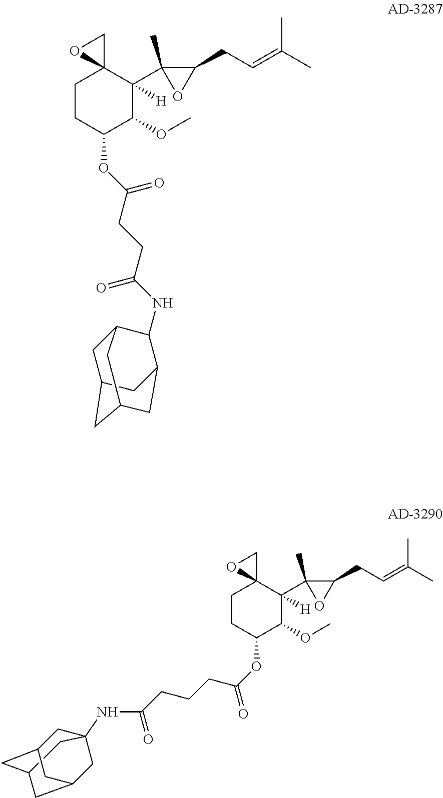 [SMILES: CO[C@@H]1[C@H](OC(=O)CCC(=O)NC2C3CC4CC(C3)CC2C4)CC[C@]2(CO2)[C@H]1[C@@]1(C)O[C@@H]1CC=C(C)C ; CO[C@@H]1[C@H](OC(=O)CCCC(=O)NC23CC4CC(CC(C4)C2)C3)CC[C@]2(CO2)[C@H]1[C@@]1(C)O[C@@H]1CC=C(C)C]

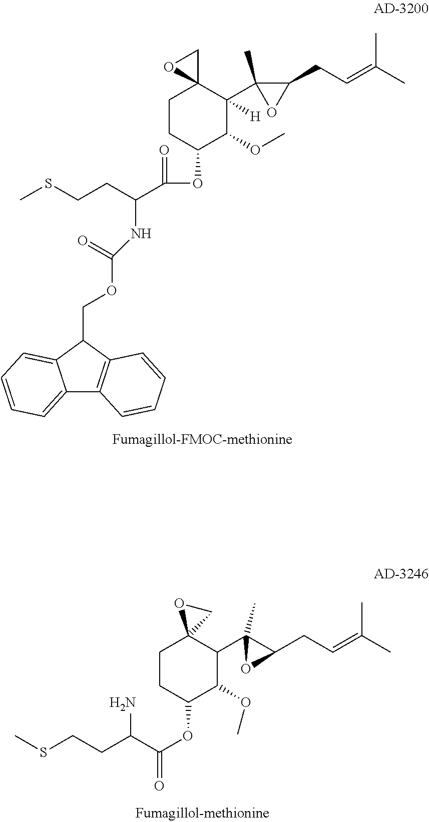

The invention further provides: Fumagillol-4-(1-adamantylamino)-4-oxobutanoate, (AD-3286); Fumagillol-4-(2-adamantylamino)-4-oxobutanoate, (AD-3287); and Fumagillol-5-(2-adamantylamino)-5-oxopentanoate, (AD-3290).

Each of the above specifically disclosed compounds constitutes a separate embodiment of the invention. Thus a compound as disclosed herein, specifically or as encompassed by a compound of Formula (I), may be used separately or in combination in preparing compositions according to the invention, or in uses and methods according to the invention. Compounds of the invention may contain one or more chiral centers. Such chiral centers may be of either the (R) or (S) configuration, or may be a mixture thereof. Thus, the compounds provided herein may be enantiomerically pure, or be stereoisomeric or diastereomeric mixtures. It is to be understood that the chiral centers of compounds provided herein may undergo epimerization in vivo. As such, one of skill in the art will recognize that administration of a compound in its (R) form is equivalent, for compounds that undergo epimerization in vivo, to administration of the compound in its (S) form.

Depending on the selection of the carbocycle R and any one or more substitution that may be present, compounds of the invention may be presented in free acid or free base form or in a form of a salt. Pharmaceutically acceptable acid addition salts may include salts derived from inorganic acids such as hydrochloric, nitric, phosphoric, sulfuric, hydrobromic, hydriodic, phosphorous, and the like, as well as the salts derived from organic acids, such as aliphatic mono- and dicarboxylic acids, phenyl-substituted alkanoic acids, hydroxy alkanoic acids, alkanedioic acids, aromatic acids, aliphatic and aromatic sulfonic acids, etc. Such salts thus include sulfate, pyrosulfate, bisulfate, sulfite, bisulfite, nitrate, phosphate, monohydrogenphosphate, dihydrogenphosphate, metaphosphate, pyrophosphate, chloride, bromide, iodide, acetate, propionate, caprylate, isobutyrate, oxalate, malonate, succinate, suberate, sebacate, fumarate, maleate, mandelate, benzoate, chlorobenzoate, methylbenzoate, dinitrobenzoate, phthalate, benzenesulfonate, toluenesulfonate, phenylacetate, citrate, lactate, maleate, tartrate, methanesulfonate, and the like. Also contemplated are salts of amino acids such as arginate and the like and gluconate, galacruronate (see, for example, Berge S. M., et al., “Pharmaceutical Salts,” J. of Pharmaceutical Science, 66:1-19 (1977)).

The acid addition salts of compounds comprising a basic functionality are prepared by contacting the free base form with a sufficient amount of the desired acid to produce the salt in the conventional manner. The free base form may be regenerated by contacting the salt form with a base and isolating the free base in the conventional manner. The free base forms differ from their respective salt forms somewhat in certain physical properties such as solubility in polar solvents, but otherwise the salts are equivalent to their respective free base for purposes of the present invention. Pharmaceutically acceptable base addition salts are formed with metals or amines, such as alkali and alkaline earth metals or organic amines. Examples of metals used as cations are sodium, potassium, magnesium, calcium, and the like. Examples of suitable amines are N,N′-dibenzylethylenediamine, chloroprocaine, choline, diethanolamine, ethylenediamine, N-methylglucamine, and procaine (see, for example, Berge S. M., et al., “Pharmaceutical Salts,” J. of Pharmaceutical Science, 66:1-19 (1977)).

Base addition salts of compounds comprising an acidic functionality may be prepared by contacting the free acid form with a sufficient amount of the desired base to produce the salt in the conventional manner. The free acid form may be regenerated by contacting the salt form with an acid and isolating the free acid in the conventional manner. The free acid forms differ from their respective salt forms somewhat in certain physical properties such as solubility in polar solvents, but otherwise the salts are equivalent to their respective free acid for purposes of the present invention.

Compounds of the invention may be formulated or provided encapsulated or contained within a nano- or a microcarrier, such as nanospheres, nanocapsules, nanoparticles, microspheres, microcapsules, microparticles etc. The carrier may be formed of any biodegradable or biostable material from which the active compound or any components encapsulated thereof may be releases in any timed fashion or at a target site. Generally, the nano- or microcarrier may be a lipid-coated nanoparticle, a protein-coated nanoparticle, or a polymer-coated nanoparticle.

In some embodiments, the carrier is composed of one or more polymers. In some embodiments, the one or more polymers is a water soluble, non-adhesive polymer. In some embodiments, polymer is polyethylene glycol (PEG) or polyethylene oxide (PEO).

In some embodiments, the polymer is polyalkylene glycol or polyalkylene oxide. In some embodiments, the one or more polymers is a biodegradable polymer. In some embodiments, the one or more polymers is a biocompatible polymer that is a conjugate of a water-soluble polymer and a biodegradable polymer. In some embodiments, the biodegradable polymer is polylactic acid (PLA), poly(glycolic acid) (PGA), or poly(lactic acid/glycolic acid) (PLGA).

In some embodiments, the carrier is or comprises PLGA. Depending on the ratio of lactide to glycolide used for the polymerization, different forms of PLGA can be selected and utilized. In some embodiments, the PLGA is selected to be of a ratio 75:25 lactic acid to glycolic acid. Other ratios such as 90:10, 80:20, 70:30, 60:40, 50:50, 40:60, 30:70, 20:80, 10:90, may be similarly prepared and used.

Compounds of the invention may be presented in compositions of a variety of forms, each selected, inter alia, based on the formulation, its intended use and mode of administration. Generally speaking, compounds of the invention may be provided in pharmaceutical acceptable forms, such as pharmaceutical compositions and formulations comprising one or more compound according to the invention and a suitable carrier.

The pharmaceutically acceptable carriers, for example, vehicles, adjuvants, excipients, or diluents, are well-known to those who are skilled in the art and are readily available to the public. It is preferred that the pharmaceutically acceptable carrier be one which is chemically inert to the active compound and one which has no detrimental side effects or toxicity under the conditions of use.

The choice of carrier will be determined in part by the particular active agent, as well as by the particular method used to administer the composition. Accordingly, there is a wide variety of suitable formulations of the pharmaceutical composition of the present invention. The following formulations are merely exemplary and are in no way limiting.

Formulations suitable for oral administration can consist of (a) liquid solutions, such as an effective amount of the compound dissolved in diluents, such as water, saline, or orange juice; (b) capsules, sachets, tablets, lozenges, and troches, each containing a predetermined amount of the active ingredient, as solids or granules; (c) powders; (d) suspensions in an appropriate liquid; and (e) suitable emulsions. Liquid formulations may include diluents, such as water and alcohols, for example, ethanol, benzyl alcohol, and the polyethylene alcohols, either with or without the addition of a pharmaceutically acceptable surfactant, suspending agent, or emulsifying agent. Capsule forms can be of the ordinary hard- or soft-shelled gelatin type containing, for example, surfactants, lubricants, and inert fillers, such as lactose, sucrose, calcium phosphate, and corn starch. Tablet forms can include one or more of lactose, sucrose, mannitol, corn starch, potato starch, alginic acid, microcrystalline cellulose, acacia, gelatin, guar gum, colloidal silicon dioxide, croscarmellose talc, magnesium stearate, calcium stearate, zinc stearate, stearic acid, and other excipients, colorants, diluents, buffering agents, disintegrating agents, moistening agents, preservatives, flavoring agents, and pharmacologically compatible carriers. Lozenge forms can comprise the active ingredient in a flavor, usually sucrose and acacia or tragacanth, as well as pastilles comprising the active ingredient in an inert base, such as gelatin and glycerin, or sucrose and acacia, emulsions, gels, and the like containing, in addition to the active ingredient, such carriers as are known in the art.

Compounds of the invention, alone or in combination with other suitable components, can be made into aerosol formulations to be administered via inhalation. These aerosol formulations can be placed into pressurized acceptable propellants, such as dichlorodifluoromethane, propane, nitrogen, and the like. They also may be formulated as pharmaceuticals for non-pressured preparations, such as in a nebulizer or an atomizer.

Compositions suitable for parenteral administration include aqueous and non-aqueous, isotonic sterile injection solutions, which can contain anti-oxidants, buffers, bacteriostats, and solutes that render the formulation isotonic with the blood of the intended recipient, and aqueous and non-aqueous sterile suspensions that include suspending agents, solubilizers, thickening agents, stabilizers, and preservatives. The compound can be administered in a physiologically acceptable diluent in a pharmaceutical carrier, such as a sterile liquid or mixture of liquids, including water, saline, aqueous dextrose and related sugar solutions, an alcohol, such as ethanol, isopropanol, or hexadecyl alcohol, glycols, such as propylene glycol or polyethylene glycol, glycerol ketals, such as 2,2-dimethyl-1,3-dioxolane-4-methanol, ethers, such as poly(ethyleneglycol) 400, an oil, a fatty acid, a fatty acid ester or glyceride, or an acetylated fatty acid glyceride with or without the addition of a pharmaceutically acceptable surfactant, such as a soap or a detergent, suspending agent, such as pectin, carbomers, methylcellulose, hydroxypropylmethylcellulose, or carboxymethylcellulose, or emulsifying agents and other pharmaceutical adjuvants.

Oils, which can be used in parenteral formulations include petroleum, animal, vegetable, or synthetic oils. Specific examples of oils include peanut, soybean, sesame, cottonseed, corn, olive, petrolatum, and mineral. Suitable fatty acids for use in parenteral formulations include oleic acid, stearic acid, and isostearic acid. Ethyl oleate and isopropyl myristate are examples of suitable fatty acid esters. Suitable soaps for use in parenteral formulations include fatty alkali metal, ammonium, and triethanolamine salts, and suitable detergents include (a) cationic detergents such as, for example, dimethyl dialkyl ammonium halides, and alkyl pyridinium halides, (b) anionic detergents such as, for example, alkyl, aryl, and olefin sulfonates, alkyl, olefin, ether, and monoglyceride sulfates, and sulfosuccinates, (c) nonionic detergents such as, for example, fatty amine oxides, fatty acid alkanolamides, and polyoxy-ethylenepolypropylene copolymers, (d) amphoteric detergents such as, for example, alkyl-aminopriopionates, and 2-alkyl-imidazoline quaternary ammonium salts, and (3) mixtures thereof.

Parenteral formulations may contain from about 0.5 to about 25% by weight of the active ingredient in solution. Suitable preservatives and buffers can be used in such formulations. In order to minimize or eliminate irritation at the site of injection, such compositions may contain one or more nonionic surfactants having a hydrophile-lipophile balance (HLB) of from about 12 to about 17. The quantity of surfactant in such formulations ranges from about 5 to about 15% by weight. Suitable surfactants include polyethylene sorbitan fatty acid esters, such as sorbitan monooleate and the high molecular weight adducts of ethylene oxide with a hydrophobic base, formed by the condensation of propylene oxide with propylene glycol. The parenteral formulations can be presented in unit-dose or multi-dose sealed containers, such as ampules and vials, and can be stored in a freeze-dried (lyophilized) condition requiring only the addition of the sterile liquid carrier, for example, water, for injections, immediately prior to use. Extemporaneous injection solutions and suspensions can be prepared from sterile powders, granules, and tablets of the kind previously described.

Compounds of the present invention may be made into injectable formulations. The requirements for effective pharmaceutical carriers for injectable compositions are well known to those of ordinary skill in the art. See Pharmaceutics and Pharmacy Practice, J. B. Lippincott Co., Philadelphia, Pa., Banker and Chalmers, eds., pages 238-250 (1982), and ASHP Handbook on Injectable Drugs, Toissel, 4th ed., pages 622-630 (1986).

Additionally, compounds of the present invention may be made into suppositories by mixing with a variety of bases, such as emulsifying bases or water-soluble bases. Formulations suitable for vaginal administration may be presented as pessaries, tampons, creams, gels, pastes, foams, or spray formulas containing, in addition to the active ingredient, such carriers as are known in the art to be appropriate.

Compounds of the invention may similarly be formulated into compositions may be used for the ocular or dermal delivery. Ocular delivery, namely delivery of a compound or a composition to an outer surface of the eye, anatomically comprising the cornea (with epithelium, bowman layer, stroma, descement membrane, endothelium), conjunctiva, and the corneo-scleral junction, i.e., the limbus, may involve presentation of a compound of the invention in a solid or a semisolid ocular insert or ocular film. The ocular film may be a solid or semisolid consistency bidimensional film designed to be placed into the conjunctival cul-de-sac or at the conjunctival surface. The ocular film may be configured for placement on the eye. The ocular insert may similarly be a solid or a semisolid device that is designed to be placed into the conjunctival cul-de-sac or at the conjunctival surface.

Ocular formulations include, but are not limited to, liquid formulations (e.g., solutions, suspensions) for topical administration as well as formulation for injection or ocular insert administration. In some cases, the ocular formulation is formulated for topical administration such as an eye drop, swab, ointment, gel, or mist (e.g., an aerosol or spray). In some cases, the formulation is an eye drop.

Dermal formulations, as well as transdermal formulations may include, for example, gels, creams, sprays, and lotions that are applied to the skin. Other formulations include patches that are affixed to the skin using an adhesive.

Compounds, compositions and formulations of the invention are suitable for administration via any desired suitable method, for example by oral (including buccal or sublingual), rectal, nasal, topical (including buccal, sublingual or transdermal), vaginal or parenteral (including subcutaneous, intramuscular, intravenous or intradermal) or any other known administration method. Each composition of the invention may be adapted or tailored for a suitable administration by formulating an effective amount of one or more of the compounds of Formula (I) with a suitable carrier, and optionally at least one additional active or non-active agent. Compositions of the invention may be administered to a human or a non-human subject to bring about prevention of treatment of a disease, which may be a topical non-systemic disease or a systemic disease.

Typically, the disease to be treated or prevented is one associated with MetAp2 activity, which a compound of the invention may be selected to partially or fully inhibit. In other words, compounds of the invention may be used in a method of blocking at least some of the biological effects of MetAp2 by binding to or interacting with MetAp2 and thereby stopping certain biological effects thereof. For instance, one of the biological effects of MetAp2 is to promote cell proliferation, and thus compounds of the invention may be used to diminish or reduce or inhibit cell proliferation.

Without wishing to be bound by theory, the mechanism of interaction between a compound of the invention and MetAp2 may involve interaction with a target molecule which may be the MetAp2 protein target, but may also be the coding gene or a gene product thereof, or a regulator protein, or a component of a signal transduction pathway comprising said gene or gene products thereof. Consequently, the specific interaction of compounds of the invention may involve either the targeting or the induction of alterations in cell function, or it may even involve both effects.

Thus, the invention further provides a method for reducing or diminishing or preventing a biological effect associated with activity of MetAp2 in a subject, the method comprising administering to said subject an effective amount of a compound of Formula (I).

In some embodiments, said reducing or diminishing or preventing comprises blocking binding to or interacting with MetAp2.

In some embodiments, the biological effect is cell proliferation.

The invention further provides a method of preventing or treating angiogenesis, an angiogenesis-related disease or an angiogenesis-dependent disease in a subject, the method comprising administering to the subject an effective amount of a compound according to Formula (I).

In some embodiments, the method is for preventing or treating angiogenesis, namely for preventing or diminishing or slowing the process involving growth of new blood vessels from pre-existing blood vessels.

In some embodiments, the method is for preventing or treating angiogenesis-related diseases, namely diseases may stem or be caused by increase neovascularization or angiogenic processes. Such diseases may include ocular angiogenesis, disease or conditions associated with ocular angiogenesis and cancer.

The cancer to be prevented or treated by use of compounds of the invention may be any malignant proliferative disease or disorder, such as blastoma, carcinoma, lymphoma, leukemia, sarcoma, mesothelioma, glioma, germinoma, choriocarcinoma, melanoma, glioblastoma, lymphoid malignancies and any other neoplastic disease or disorder. In some embodiments, the cancer that can be prevented or treated using compounds of the invention include, but are not limited to, squamous cell cancer (e.g., epithelial squamous cell cancer), lung cancer including small-cell lung cancer, non-small cell lung cancer, adenocarcinoma of the lung and squamous carcinoma of the lung, cancer of the peritoneum, hepatocellular cancer, gastric or stomach cancer including gastrointestinal cancer, pancreatic cancer, glioblastoma, cervical cancer, ovarian cancer, liver cancer, bladder cancer, hepatoma, breast cancer, colon cancer, rectal cancer, colorectal cancer, endometrial or uterine carcinoma, salivary gland carcinoma, kidney or renal cancer, prostate cancer, vulval cancer, thyroid cancer, hepatic carcinoma, anal carcinoma, penile carcinoma, melanoma as well as head and neck cancer. Solid cancers may include, for example, breast cancer, prostate cancer, sarcomas, and skin cancer.

In some embodiments, the method of the invention is for preventing or treating angiogenesis-dependent diseases such as ocular neovascular diseases, wounds, chronic ulcer, ischemic stroke, myocardial infarction, angina pectoris, peripheral artery disease, critical limb ischemia, diabetic foot ulcer, and cerebrovascular dementia.

The invention further provides a method for preventing or treating a cancer in a subject, the method comprising administering to the subject an effective amount of a compound of Formula (I).

The invention further provides a method for preventing or treating a pulmonary and hepatic fibrosis in a subject, the method comprising administering to the subject an effective amount of a compound of Formula (I).

In some embodiments, the fibrosis is selected from formation of a scar tissue or a tissue lesion causing chronic and progressive impairment of an organ in a subject's body.

The invention further provides a method for preventing or treating a disease in a subject, the method comprising administering to the subject an effective amount of a compound of Formula (I), wherein the disease is selected from angiogenesis, ocular angiogenesis, ocular neovascular diseases, wounds, chronic ulcer, ischemic stroke, myocardial infarction, angina pectoris, peripheral artery disease, critical limb ischemia, diabetic foot ulcer, cerebrovascular dementia, cancer, pulmonary fibrosis, hepatic fibrosis, endometriosis, arthritis (e.g., rheumatoid arthritis), autoimmune diseases, obesity and microsporidiosis.

The invention further provides methods of preventing or treating at least one ocular or dermal disease or condition in a subject, the disease or condition being associated with MetAp2 activity, the methods comprising ocular or dermal delivery of a composition comprising at least one MetAp2 inhibitor according to the invention to the subject.

In some embodiments of compositions and methods of the invention, the compound of Formula (I) is a compound selected from compounds herein designated: AD-3281, AD-3306, AD-3201 and AD-3306.

The term “treatment” as used herein encompasses both prophylaxis and treatment of a disease, disorder or condition. The term generally refers to administering a therapeutic amount of a compound or a composition comprising the compound, which amount is effective to ameliorate undesired symptoms associated with a disease, to prevent the manifestation of such symptoms before they occur, to slow down the progression of the disease, slow down the deterioration of symptoms, to enhance the onset of remission period, slow down the irreversible damage caused in the progressive chronic stage of the disease, to delay the onset of said progressive stage, to lessen the severity or cure the disease, to improve survival rate or more rapid recovery, or to prevent the disease form occurring or a combination of two or more of the above

The “effective amount” of a compound or a composition comprising the compound, for purposes herein, is determined by such considerations as may be known in the art. The amount must be effective to achieve the desired therapeutic effect as described above, depending, inter alia, on the type and severity of the disease to be treated and the treatment regime. The effective amount is typically determined in appropriately designed clinical trials (dose range studies) and the person versed in the art will know how to properly conduct such trials in order to determine the effective amount. As generally known, an effective amount depends on a variety of factors including the affinity of the ligand to the receptor, its distribution profile within the body, a variety of pharmacological parameters such as half-life in the body, on undesired side effects, if any, on factors such as age and gender, etc.

The invention further provides a kit comprising a compound of the invention and instructions of use.

### BRIEF DESCRIPTION OF THE DRAWINGS

In order to better understand the subject matter that is disclosed herein and to exemplify how it may be carried out in practice, embodiments will now be described, by way of non-limiting example only, with reference to the accompanying drawings, in which:

FIGS. 1A-G demonstrate the inhibitory effect of compounds of the invention on MetAp2. Compound AD-3201 is TNP-470.

FIGS. 2A-C depict the overall effect of the different compounds on HUVEC.

FIG. 3 demonstrates mouse laser induce CNV. Compound AD3281 led to 46% in the selected dose (12 μg per eye). Eylea lead to 59% in the given dose and time.

FIGS. 4A-D show the chemical synthesis and Characterization of AD-3281. A. Schematic synthesis of AD-3281, under basic conditions Fumagillin, converted to fumagilol and AD-3281 was then achieved after an esterification reaction. B. Mass spectrometry (MS) of AD-3281 C. H NMR spectra of AD-3281. D. HPLC for AD-3281 and TNP-470, early peak present TNP-470, pink peak state a retention time of ˜15 min for AD-3281. The detection was at 205 nm.

FIGS. 5A-B demonstrate basal MetAp1 and MetAp2 expression in endothelial and cancer cells. (A, B) Western blot analyses for determining the expression of (A) MetAp2 and (B) MetAp1in HUVECs, A375, and MDA-MB-231. MetAp2 levels were found to be higher in cancer cells than the levels in HUVECs. n=3.

FIGS. 6A-B demonstrate how AD-3281 inhibits the activity of MetAp2 in enzymatic assay. A. AD-3281 affects the enzymatic activity of rhMetAp2 and reduced the activity of MetAp2 in cancer cells. L-Met-AMC was added as a substrate to test the enzymatic activity of MetAp2. The addition of 2.5 μM AD-3281 inhibits rhMetAp2's enzymatic activity by 95%, and by 64% and 80% in A375 and MDA-MB-231 cancer cells respectively over the course of 1 h. B. Measurement of MetAp2's activity after 10 min. AD-3281 showed a reduction in MetAp2 activity compared with the control group. n=3. *p<0.05, **P<0.01 Results are presented as mean±SEM.

FIGS. 7A-E depict the effect of AD-3281 on endothelial and cancer cells function. (A, B) AD-3281 impairs the ability of cells to proliferate. Cells were treated with different concentrations of AD-3281 for 72 h after which an MTT assay was conducted to quantify their proliferation. Compared with control dose-dependent reduction is observed. A. HUVECs n=6 B. A375 n=7 (C, D) Effect of increasing concentration of encapsulated AD-3281 on cell viability. C. A375 left 72 h, right 96 h of incubation D. HUVECs. n=5 E. AD-3281 affects A375 cell viability in 3D compared with non-treated control cells, cell viability measured using WST1 assay after the incubation of 96 h n=4. *p<0.05, **P<0.01, *** P<0.001. Results are presented as mean±SEM.

FIGS. 8A-H show how AD-3281 suppresses A375 and MDA-MB-231 xenograft growth in treated mice. (A, B) AD-3281 affects the volume of tumors in treated groups compared with the control group, A. S.C. injected mice with A375 were treated with 15 mg/kg and 30 mg/kg every other day n=7 B. effect of AD-3281 on MDA-MB-231 tumors treated with 7.5 mg/kg and 15 mg/kg every other day. n=4 (C, D) The mean weight of the tumors extracted from treated and untreated mice C. A375, D. MDA-MB-231. E. Representative images of tumor extracted from the untreated and treated mice following 18 and 15 days of treatment in mice injected with A375 and MDA-MB-231 respectively. F. Immunofluorescent staining for MetAp2 (red), CD31 (green), and DAPI (blue) G. histology of extracted tumors from treated and untreated mice stained with H&E. H. Immunofluorescent staining for Ki67 (green) and DAPI (blue). *p<0.05, **P<0.01, *** P<0.001. Results are presented as mean±SEM. Scale bar 100 μm.

FIGS. 9A-E depict PLGA nanoparticles preparation and characterization. A. Illustration of the synthesis process of the encapsulated AD-3281 in PLGA nanoparticles by emulsification-evaporation method (B) TEM images of PLGA nanoparticles (C) Zeta potential of nanoparticles (D) Typical DLS measurements of PLGA nanoparticles; the graphs present. (E) summary of formulation measured parameters.

FIGS. 10A-B show basal MetAp2 expression and enzymatic activities in endothelial and cancer cells in response to treatments. (A) Western blot analyses for determining the expression of MetAp2 HUVECs, A375, and MDA-MB-231. MetAp2 levels were found to be higher in cancer cells than the levels in HUVECs. n=3. (B) AD-3281 affects the enzymatic activity of rhMetAp2 and reduced the activity of MetAp2 in cancer cells. L-Met-AMC was added as a substrate to test the enzymatic activity of MetAp2. The addition of 2.5 M AD-3281 inhibits rhMetAp2's enzymatic activity by 95%, and by 64% and 80% in A375 and MDA-MB-231 cancer cells respectively over the course of 1 h. In the bottom, measurement of MetAp2's activity after 10 min of reaction. AD-3281 showed a reduction in MetAp2 activity compared with the control group. n=3. *p<0.05, **P<0.01 Results are presented as mean±SEM.

FIG. 11 depicts MetAp1 basal protein expression. Western blot analysis of MetAp1 from cell lysis and quantification of the obtained signal.

FIGS. 12A-B show how AD-3281 inhibits the activity of MetAp2 in enzymatic assay. (A) AD-3281 suppressed HUVEC and A375 cell proliferation. Cells were treated with different concentrations of AD-3281 for 72 h after which an MTT assay was conducted to quantify their proliferation. Compared with control dose-dependent reduction is observed. n=6-7. (B) representative microscopic bright field images of HUVEC tube formation (converted to black and white) showing impaired tubes in 100 μM AD-3281 culture.

FIG. 13 depicts HUVEC tube formation. Examples of images obtain by brightfield microscope AD-3281 treated HUVECs were allowed to form tube formation and images were taken after 12 h and compared to untreated control.

FIGS. 14A-B show how AD-3281 suppresses A375 and MDA-MB-231 xenograft growth in treated mice. (A) S.C. injected mice with A375 were treated with 15 mg/kg and 30 mg/kg every other day n=7 Graph shows the mean weight of the tumors extracted from treated and untreated mice and in the bottom resected tumors at day 18 are presented (B) Effect of AD-3281 on MDA-MB-231 tumors treated with 7.5 mg/kg and 15 mg/kg every other day. n=4. Graph shows tumor weight at the end point and in the bottom representative images of tumor extracted from the untreated and treated mice following 15 days of treatment.

FIGS. 15A-C show Immunofluorescent staining of resected A375 xenografts treated with 30 mg/kg q.o.d. (a) Sections were reacted with antibodies against MetAp2 (red), CD31 (green), and DAPI (blue). (b) Immunofluorescent staining for Ki67 (green) and DAPI (blue). (c). Histology of extracted tumors from treated and untreated mice stained with H&E.

FIG. 16 depicts Immunofluorescent staining for MDA-MB-231 resected tumors (7 kg/mg). MetAp2 (red), CD31 (green), and DAPI (blue). Scale bar 100 μm.

FIGS. 17A-E depict PLGA nanoparticles preparation and characterization. (a) Illustration of the synthesis process of the encapsulated AD-3281 in PLGA nanoparticles by emulsification-evaporation method (b) TEM images of PLGA nanoparticles (c) Zeta potential of nanoparticles (d) Typical DLS measurements of PLGA nanoparticles; the graphs present. (e) summary of formulation measured parameters.

FIGS. 18A-C PLGA nanoparticles cell uptake. A375 cells were exposed to encapsulate 6-coumarin in PLGA nanoparticles for 0 min, 4, 7 h (a) FACS dot plot analysis showing cells population that contain nanoparticles labeled with 6-coumarin. Column plot show normalized average uptake of fluorescent nanoparticles. n=6. (b) MTT proliferation assays performed in A375 after 72 h and 96 h post exposure to encapsulated AD-3281 (c) Viability of 3D spheroids post exposure to free or encapsulated AD-3281 as measured with WST-8 reagent.

FIG. 19 shows ERG study in mice showing no damage to vision using Low dose (LD): 12 μg per eye and High dose (HD): 48 μg per eye of AD328.

### DETAILED DESCRIPTION OF EMBODIMENTS

Fumagillol-1-adamantylacetate (AD-3281)—Procedure A 100 mg Fumagillol (350 μmol) were dissolved in 30 ml Dichloromethane in a 50 ml round bottom flask. 68 mg (350 μmol) 1-adamantane acetic acid were added followed by 22 mg (180 μmol) 4-(dimethylamino)pyridine (DMAP). The solution was magnetically stirred and 96 mg (0.5 mmol) N-(3-Dimethylaminopropyl)-N′-ethylcarbodiimide hydrochloride (EDC HCl) were added. The solution was left to stir for 8 h. To the mixture 100 ml Dichloromethane and 25 ml Methanol were added and the compound was washed in a separatory funnel with 0.1N HCl (2 times) then 0.1N Sodium bicarbonate (2 times) and then with water. The resulted solution was dried on MgSO4 filtered and evaporated to dryness. Then, purified by column chromatography on silica gel using Dichloromethane with increasing concentrations of Methanol.

Fumagillol-1-adamantylacetate (AD-3281)—Procedure B

In a 50 ml round bottom flask 57 mg Fumagillol (200 μmol) were dissolved in 25 ml Dichloromethane. 220 μmol carboxyl bulky derivative was added followed by 10 mg (80 μmol) 4-(dimethylamino)pyridine (DMAP). The solution was magnetically stirred and 80 mg (400 μmol) N-(3-Dimethylaminopropyl)-N′-ethylcarbodiimide hydrochloride (EDC HCl) were added. The solution was left to stir during 8 h. To the mixture 100 ml Dichloromethane and 25 ml Methanol were added and the compound was washed in a separatory funnel with 0.1N HCl (2 times) then 0.1N Sodium bicarbonate (2 times) and then with water. The resulted solution was dried on MgSO4 filtered and evaporated to dryness. Then, purified by column chromatography on silica gel using Dichloromethane with increasing concentrations.

Fumagillol-1-adamantylacetate (AD-3281)—Procedure C

The synthesis of AD-3281 initiated by fumagillol synthesis: 5 mmole Fumagillin (dicyclohexylamine salt), was dissolved in 200 ml Ether in a 1000 ml round bottom flask. 200 ml NaOH (1N) were added and the solution was magnetically stirred overnight at room temperature. The reaction solution was transferred to a separatory funnel and the phases were separated. The upper layer ether phase was washed 3 times with 100 ml Brine. Dried on MgSO₄ and evaporated to dryness. Synthesis of Fumagillol-1-adamantylacetate (AD-3281) was carried out as follows: 1.0 mmole Fumagillol and 1.2 mmole 1-Adamantaneacetic acid were dissolved in 30 ml Dichloromethane in a 50 ml round bottom flask. To the stirred solution 0.6 mmole) 4-(Dimethylamino) pyridine was added followed by 3 mmole N-(3-Dimethylaminopropyl)-N′-ethyl carbodiimide hydrochloride. The mixture was stirred overnight and then transferred to a separatory funnel. 70 ml Dichloromethane and 50 ml Methanol were added. The solution was washed 2 times with 50 ml HCl (0.1 N), 2 times with 50 ml Sodium Bicarbonate (0.1 N), and then with 50 ml water. Dried on MgSO₄ and evaporated to dryness. The residue was purified on a silica gel column using Dichloromethane: Methanol gradient.

Fumagillol-1-adamantylcarboxylate (AD-3283)

This compound was prepared from 200 μmol Fumagillol and 220 μmol, 1-adamantylcarboxylic acid using general procedure A.

Fumagillol-4-(1-adamantylamino)-4-oxobutanoate (AD-3286)

This compound was prepared from 200 μmol Fumagillol and 220 μmol 4-(1-adamantylamino)-4-oxobutanoic acid, using general procedure A.

Fumagillol-4-(2-adamantylamino)-4-oxobutanoate (AD-3287)

This compound was prepared from 100 μmol Fumagillol and 110 μmol 4-(2-adamantylamino)-4-oxobutanoic acid, using general procedure A.

Fumagillol-{[(2-adamantylamino)carbonothioyl]amino}acetate (AD-3295)

This compound was prepared from 200 μmol Fumagillol and 220 μmol {[(2-adamantylamino)carbonothioyl]amino}acetic acid, using general procedure A.

Fumagillol-6-{[(1-adamantylamino)carbonothioyl]amino}hexanoate (AD-3294)

This compound was prepared from 200 μmol Fumagillol and 220 μmol {[(2-adamantylamino)carbonothioyl]amino}hexanoic acid, using general procedure A.

Fumagillol-5-(2-adamantylamino)-5-oxopentanoate (AD-3290)

This compound was prepared from 200 μmol Fumagillol and 220 μmol (2-adamantylamino)-5-oxopentanoic acid, using general procedure A.

Fumagillol-(3-bromo-1-adamantyl)acetate (AD-3301)

This compound was prepared from 200 μmol Fumagillol and 220 μmol (3-bromo-1-adamantyl)acetic acid, using general procedure A.

Fumagillol-(3,5-dimethyl-1-adamantyl)acetate (AD-3302)

This compound was prepared from 200 μmol Fumagillol and 220 μmol (3,5-dimethyl-1-adamantyl)acetic acid, using general procedure A.

Fumagillol-bicyclo[2.2.1]hept-5-ene-2-carboxylate (AD-3303)

This compound was prepared from 200 μmol Fumagillol and 220 μmol bicyclo[2.2.1]hept-5-ene-2-carboxylic acid, using general procedure A.

Fumagillol-chloro(3,5,7-trimethyl-1-adamantyl)acetate (AD-3305)

This compound was prepared from 200 μmol Fumagillol and 220 μmol chloro(3,5,7-trimethyl-1-adamantyl)acetic acid using general procedure A.

Fumagillol-3,5,7-trimethyladamantane-1-carboxylate (AD-3306)

This compound was prepared from 200 μmol Fumagillol and 220 μmol 3,5,7-trimethyladamantane-1-carboxylic acid, using general procedure A.

### Activity Enzymatic Inhibition Assay

Quantitative MetAP2 activity assay by N-terminal cleavage of substrate 7-amino-4-methyl-courmarin was performed as follows: The activity of MetAP2 was measured using fluorescent peptide substrate L-Met-AMC (Santa Cruz Biotechnology, Inc, sc-207807) as described by Garrabrant et al. (Angiogenesis 7: 91-96, 2004). Briefly, the reaction (100 μl final) was initiated by adding 0.5 μg of MetAP2 or 5 μg HUVEC homogenate to reaction buffer containing 50 mM HEPES (pH 7.4), 100 mM NaCl, 0.1 mM CoCl2, 1 mg/ml PEG6000, and 250 μM L-Met-AMC substrate. The assay was prepared in black flat-bottomed 96-well microtiter plates and fluorescence was measured every 20 s over a period of 60 min at 25° C. using a microplate fluorometer (Bioteck, SynergyHT), with excitation and emission wavelengths set at 360 and 440 nm respectively. As a control the inhibition of the enzyme was obtained by incubating it with TNP-470 for 15 min before the addition of the substrate.

### HUVEC Proliferation Assay

Human Umbilical Vein Endothelial Cells (HUVECs) or human melanoma cell line (A375) were used to validate the ability of the compound to suppress endothelial cell proliferation. Each 1500 A375 cells/well or 2000 HUVEC cells/well were seeded in a 96 wells plate and allowed to adhere to the plate for few hours. Compounds were then added to the cells at concentration range of 0.05-10 μM (<0.1% DMSO). Cells were then incubated for additional 72 hours and MTT was done (2 to 3 h at 37° C.). The dye was solubilized with DMSO and the absorbance was determined at 570 nm.

Compounds mentioned herein, amongst them compounds of the invention, include:

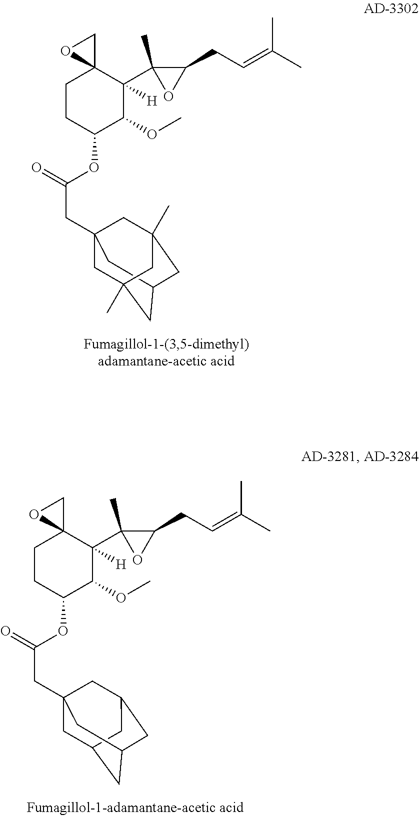 [SMILES: CO[C@@H]1[C@H](OC(=O)CC23CC4CC(C)(CC(C)(C4)C2)C3)CC[C@]2(CO2)[C@H]1[C@@]1(C)O[C@@H]1CC=C(C)C ; CO[C@@H]1[C@H](OC(=O)CC23CC4CC(CC(C4)C2)C3)CC[C@]2(CO2)[C@H]1[C@@]1(C)O[C@@H]1CC=C(C)C]

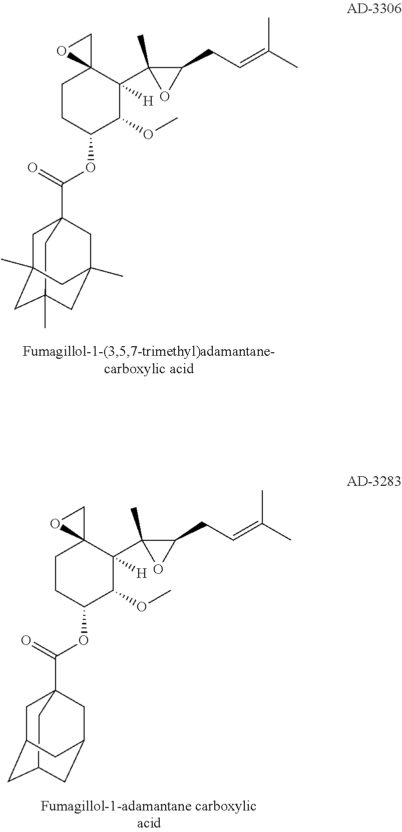 [SMILES: CO[C@@H]1[C@H](OC(=O)C23CC4(C)CC(C)(CC(C)(C4)C2)C3)CC[C@]2(CO2)[C@H]1[C@@]1(C)O[C@@H]1CC=C(C)C ; CO[C@@H]1[C@H](OC(=O)C23CC4CC(CC(C4)C2)C3)CC[C@]2(CO2)[C@H]1[C@@]1(C)O[C@@H]1CC=C(C)C]

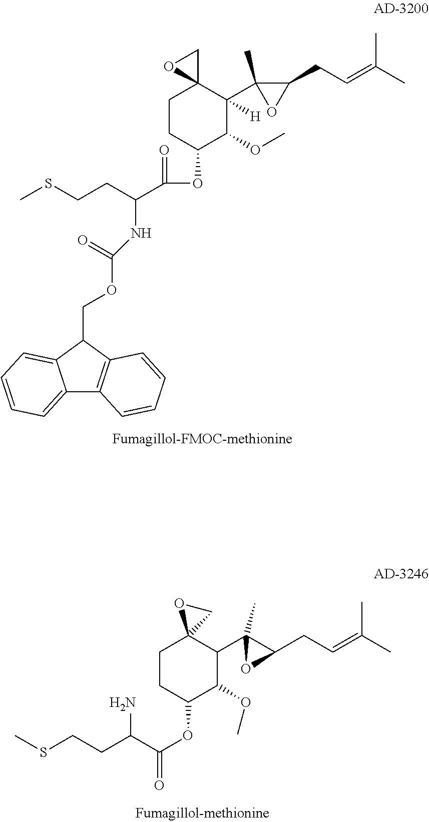

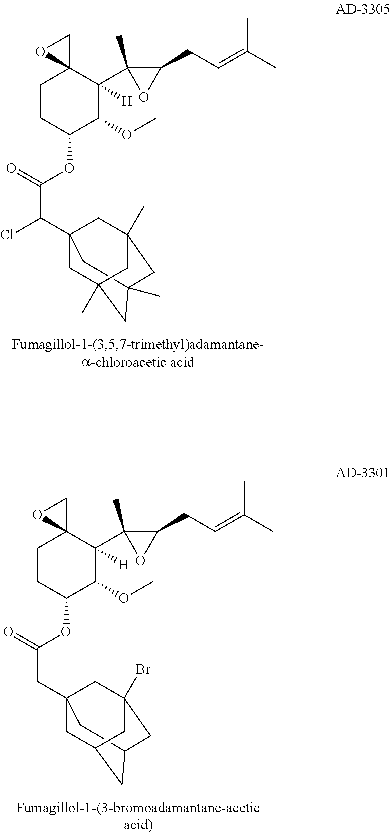 [SMILES: CO[C@@H]1[C@H](OC(=O)C(Cl)C23CC4(C)CC(C)(CC(C)(C4)C2)C3)CC[C@]2(CO2)[C@H]1[C@@]1(C)O[C@@H]1CC=C(C)C ; CO[C@@H]1[C@H](OC(=O)CC23CC4CC(CC(Br)(C4)C2)C3)CC[C@]2(CO2)[C@H]1[C@@]1(C)O[C@@H]1CC=C(C)C]

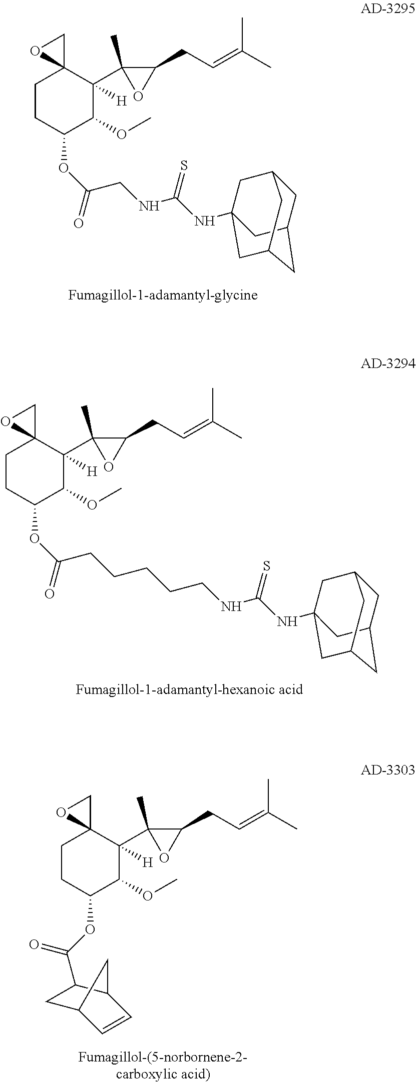 [SMILES: CO[C@@H]1[C@H](OC(=O)CNC(=S)NC23CC4CC(CC(C4)C2)C3)CC[C@]2(CO2)[C@H]1[C@@]1(C)O[C@@H]1CC=C(C)C ; CO[C@@H]1[C@H](OC(=O)CCCCCNC(=S)NC23CC4CC(CC(C4)C2)C3)CC[C@]2(CO2)[C@H]1[C@@]1(C)O[C@@H]1CC=C(C)C ; CO[C@@H]1[C@H](OC(=O)C2CC3C=CC2C3)CC[C@]2(CO2)[C@H]1[C@@]1(C)O[C@@H]1CC=C(C)C]

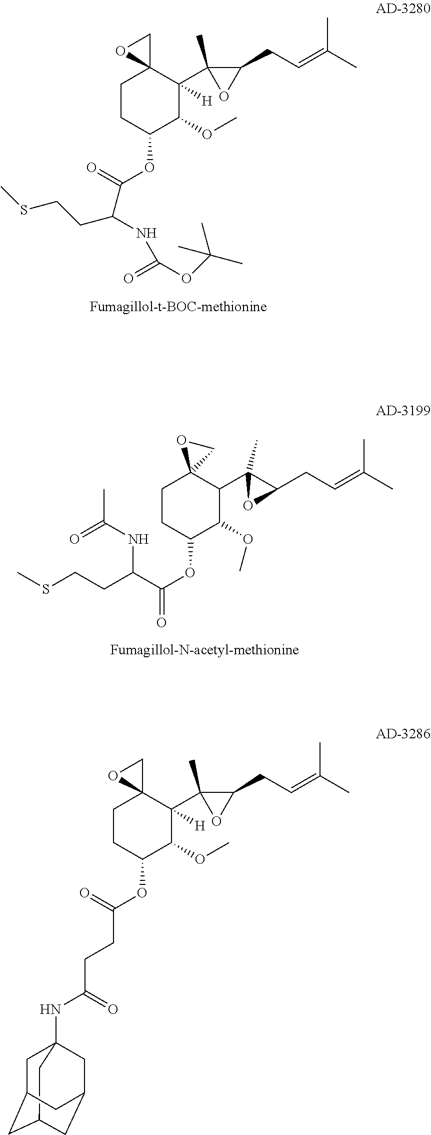

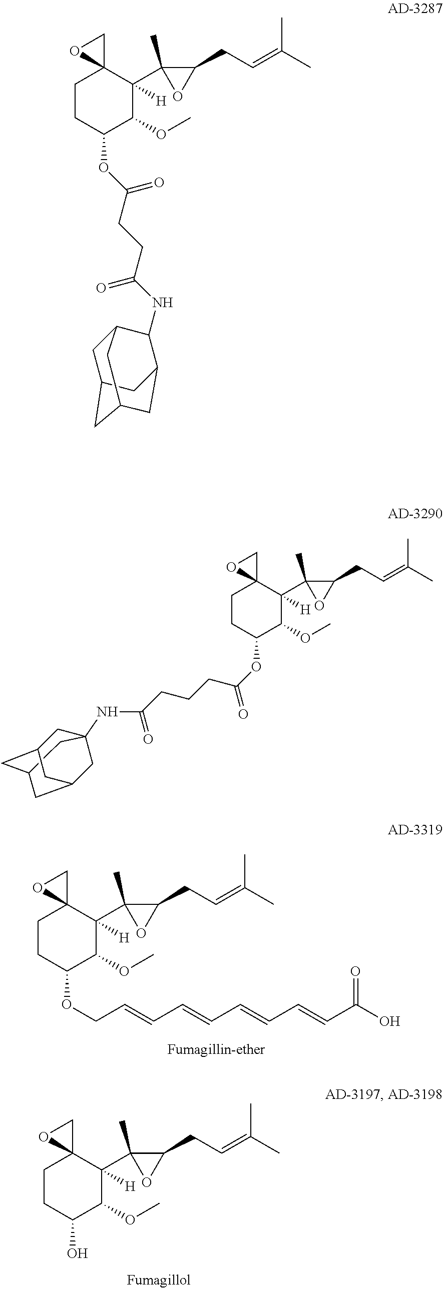 [SMILES: CO[C@@H]1[C@H](OC(=O)CCC(=O)NC2C3CC4CC(C3)CC2C4)CC[C@]2(CO2)[C@H]1[C@@]1(C)O[C@@H]1CC=C(C)C ; CO[C@@H]1[C@H](OC(=O)CCCC(=O)NC23CC4CC(CC(C4)C2)C3)CC[C@]2(CO2)[C@H]1[C@@]1(C)O[C@@H]1CC=C(C)C ; CO[C@@H]1[C@H](OC/C=C/C=C/C=C/C=C/C(=O)O)CC[C@]2(CO2)[C@H]1[C@@]1(C)O[C@@H]1CC=C(C)C ; CO[C@@H]1[C@H](O)CC[C@]2(CO2)[C@H]1[C@@]1(C)O[C@@H]1CC=C(C)C]

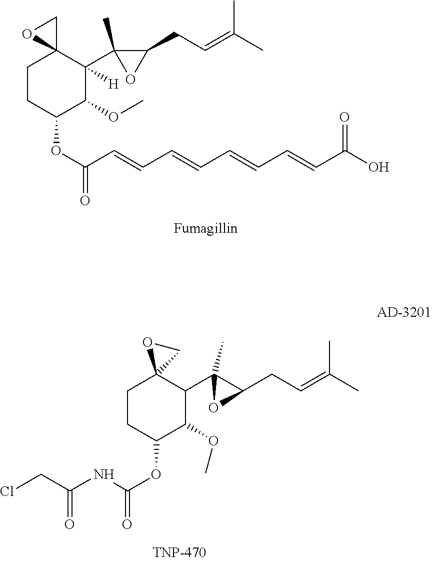

### Experimental Set-Up 1

### Enzymatic Assays

The inhibitory effect of the compounds were tested in MetAp2 enzymatic assay.

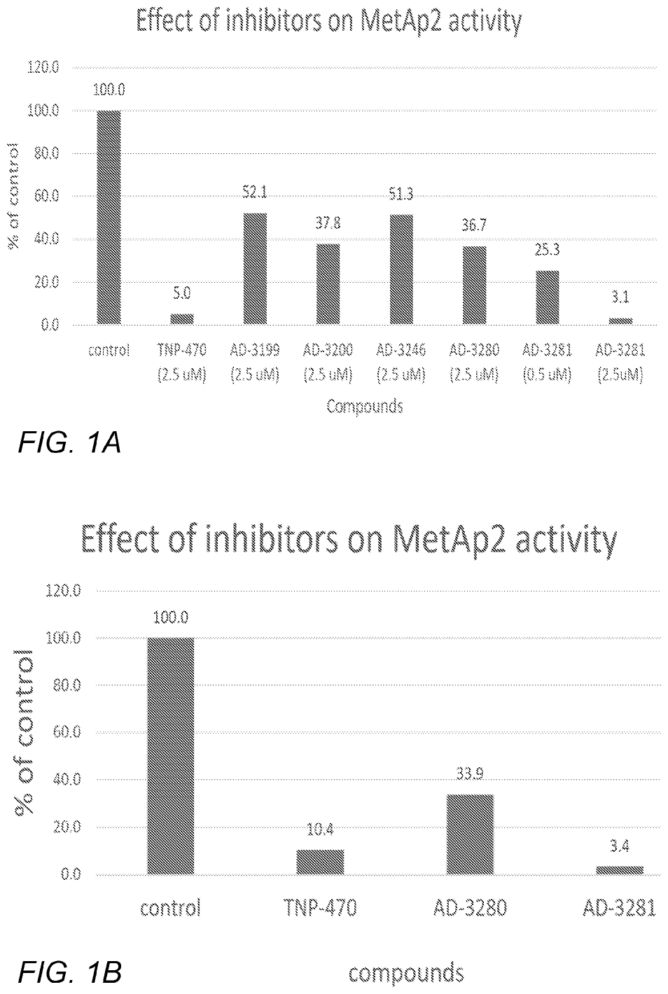

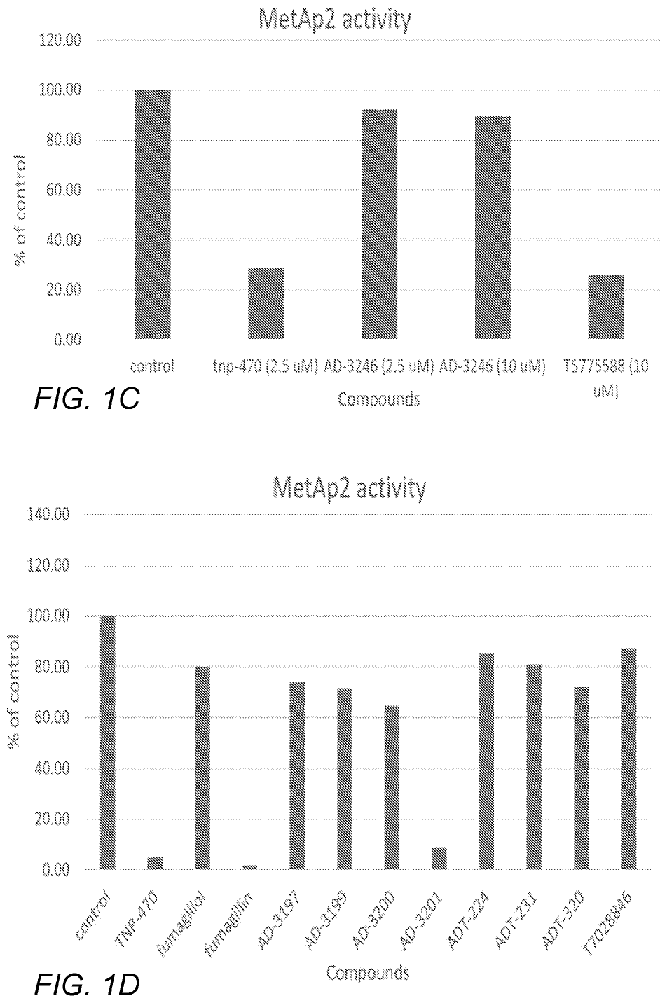

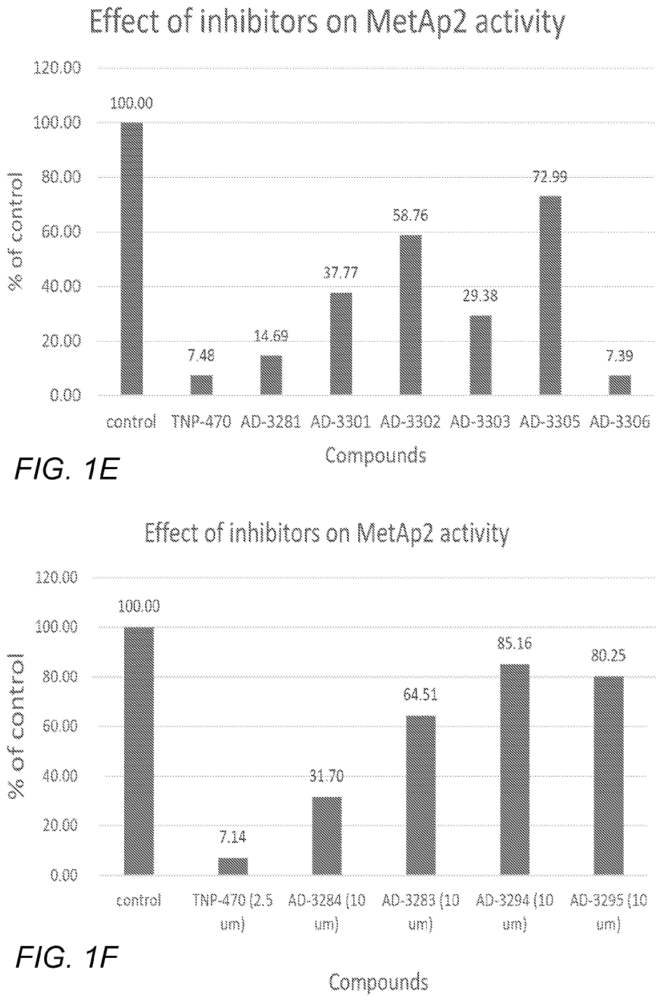

The most active compounds were compounds herein numbered AD-3281 and AD-3306 (FIGS. 1A-B, E-G).

Compound AD-3201 showed similar (and slightly better) activity compared to TNP-4′70 in inhibiting the catalytic activity of MetAp2. Concentration of 0.5 microM inhibited 75% of the activity and 2.5 microM yielded 97% inhibition (95% with TNP-470). Compound AD-3306 was almost identical to TNP-470 (FIGS. 1C-D).

### Cell Assays Results

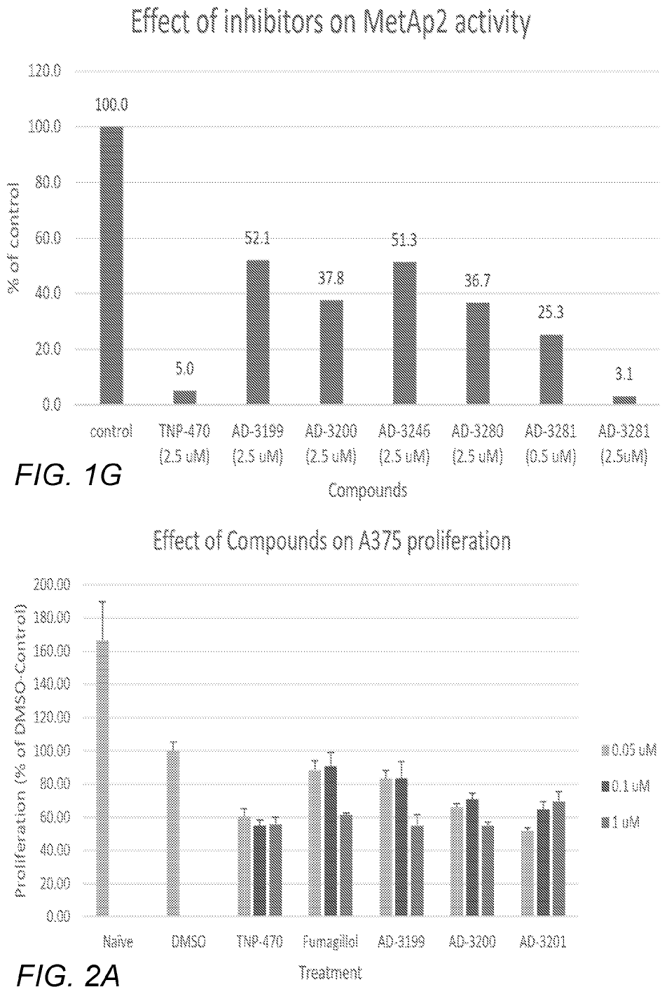

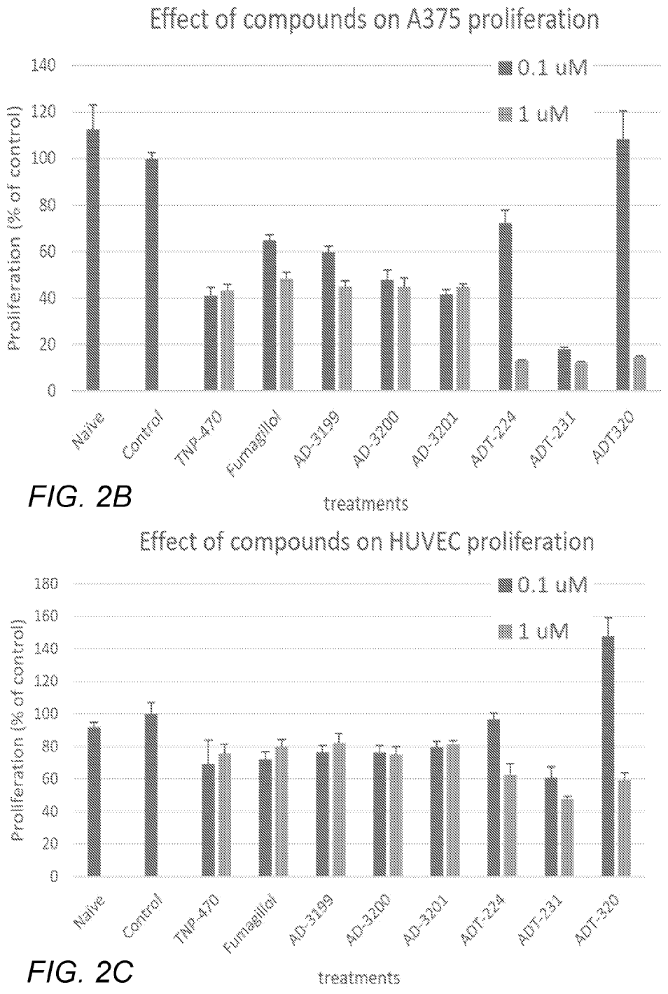

The capability to suppress cell proliferation in endothelial and cancer cells was evaluated using the MTT assay. The overall effect of the different compounds on HUVEC and A375 viability was relatively similar to TNP-470 and fumagillol (FIGS. 2A-C).

### Laser Induced CNV Using AD3281 in Comparison to EYLEA

Methionine Aminopeptidase 2 (MetAp2) is an intracellular enzyme that is overexpressed in activated endothelium. We previously demonstrated that MetAp2 can be used as a target to regress Choroidal Neovascularization (CNV) in mice (1). Biochemical inhibition of MetAp2 can be done with small molecules. Recently, we identified another novel MetAp2 inhibitor, AD3281, which show comparable efficacy to TNP-470. In this work, we evaluated the new MetAp2 inhibitor in comparison to Eylea which is the standard of care, as treatment for “wet” AMD. To further stabilize and solubilize the compounds intravitreally.

### Method

Laser-induced CNV was generated by a previously described technique with some modifications. C57Bl/6J mice (6-8 weeks) were anesthetized by intraperitoneal injections of a mixture of 85% ketamine and 15% xylazine. A mixture of 0.5% tropicamide and 0.5% phenylephrine hydrochloride was applied to both eyes to dilate the pupils. Lesions were induced by a diode pumped solid state laser (0.1 s; spot size, 100 μm; power 120 mW) around the optic nerve through a slit lamp delivery system using a Nidek while a hand-held cover slide was used as a contact lens. Only lesions in which a subretinal bubble or focal serous detachment of the retina developed were used for the experiments. For this purpose, 4 burns were performed per eye while leaving a space around the optic disc. Intravitreal injections of the treatments were performed using a PLI-100 Pico-Injector following the laser procedure. After 7 days mice were euthanized and their eyes were removed and fixed in 4% paraformaldehyde for 60 min. The cornea and lens were removed, and the entire retina was carefully dissected from the eyecup Choroid.

Blood vessels were labeled using a 1:200 dilution of isolectin IB4 conjugated with Alexa Fluor 488 or lectin-FITC. The eyecup was flat-mounted in an aqua-mount with the sclera facing down and the choroid facing up. Fluorescent images of choroidal flat-mounts were captured. The CNV area (presented in μm2) in choroidal flat mount was evaluated using ImageJ software

Concentration of compounds: 12 mg/ml (26 mM), PBS 0.2% solutol 30% hydroxypropyl beta cyclodextrin.

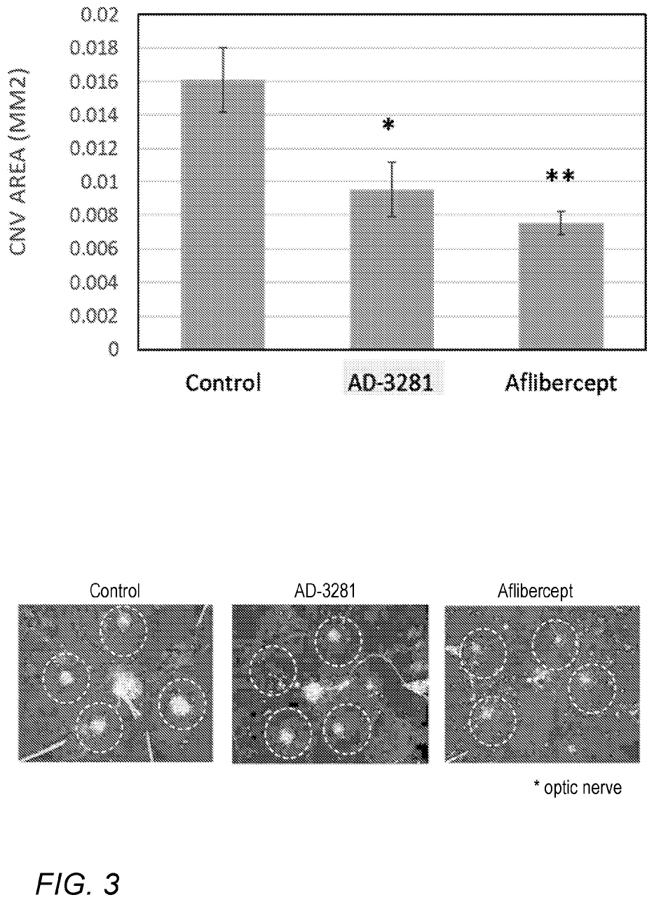

Results are shown in FIG. 3.

### Synthesis of AD-3281

The synthesis of fumagillol derivate was done as follows: 5.0 mmole Fumagillin (dicyclohexylamine salt), was dissolved in 200 ml Ether in a 1000 ml round bottom flask. 200 ml NaOH (1N) were added and the solution was magnetically stirred overnight at room temperature. The reaction solution was transferred to a separatory funnel and the phases were separated. The upper layer ether phase was washed 3 times with 100 ml Brine. Dried on MgSO₄ and evaporated to dryness. 1.0 mmole Fumagillol and 1.2 mmole 1-Adamantaneacetic acid were dissolved in 30 ml Dichloromethane in a 50 ml round bottom flask. To the stirred solution 0.6 mmole) 4-(Dimethylamino) pyridine was added followed by 3.0 mmole N-(3-Dimethylaminopropyl)-N′-ethyl carbodiimide hydrochloride. The mixture was stirred overnight and then transferred to a separatory funnel. 70 ml Dichloromethane and 50 ml Methanol were added. The solution was washed 2 times with 50 ml HCl (0.1 N), 2 times with 50 ml Sodium Bicarbonate (0.1 N), and then with 50 ml water. Dried on MgSO₄ and evaporated to dryness. The residue was purified on a silica gel column using Dichloromethane: Methanol gradient.

### Characterization of AD-3281

H¹-NMR (CDCl₃) spectrometer analysis (Bruker 500 MH, Hebrew University of Jerusalem, service of the core facility) was conducted to determine the molecular structure of the novel inhibitor.

### High-Performance Liquid Chromatography (HPLC) for AD-3281 Determination

AD-3281 was detected as a peak using HPLC (System Gold Microbore, Beckman Coulter) at 15 min with 50% ACN in water. The Flow rate was 1 ml/min and injection volume of sample 10 μL into a Kinetex 5u EVO Column (C18, 150×4.6 mm). The temperature was set to 20° C. and the detection was monitored at 205 nm wavelength.

### Cell Culture

Human umbilical vein endothelial cells were purchased from Lonza (Walkersville, MD, USA) and were grown in PeproGrow endothelial cell media supplemented with MacroV with 1% penicillin/streptomycin. MDA-MB-231 and A375 were purchased from ATCC and were maintained in Dulbecco's modified eagle's medium (DMEM), supplemented with 10% fetal calf serum (FCS) media with 1% penicillin/streptomycin. Cells kept in humidified incubated at 37° C. with 5% C₀₂. All cells were characterized before use, mycoplasma-free, using an EZ-PCR Mycoplasma Test Kit (Biological Industries).

### Western Blot

Cells extracts were isolated using RIPA buffer with protease inhibitor cocktail (Sigma, S8820) for 30 min on ice. Lysates were centrifuged, and the supernatant was collected. Using BCA Protein Assay kit (Pierce™, Thermo Fisher Scientific, Cambridge, MA, USA) isolated protein content was determined. Proteins (15 μg protein) were separated by a 12.5% Tris-glycine SDS-PAGE and transferred onto a Polyvinylidene difluoride membrane (Millipore Corporation, Billerica, MA, USA). The Membranes were incubated in blocking buffer for 2 h and then incubated with anti-MetAp2 abs or anti-MetAp1 abs, Ab134124 or Ab185540 (Abcam, Cambridge, UK), respectively, overnight at 4° C. in TBST containing 5% BSA. After washing three times for 5 min in TBST, the membranes were incubated with a 1:5000 dilution of goat anti-rabbit secondary ab conjugated to horseradish peroxidase for 1 h (Ab97080, Abcam). 0-actin or cofilin, Ab49900 or Ab124979 (Abcam), respectively, were used as the loading control.

### MetAp2 Enzymatic Activity Assays

A375 and MDA-MB-231 were sub cultivated using trypsin, then centrifuge and counted. To obtain a homogenate containing an equivalent cell number, cells were resuspended in an appropriate volume of cold RIPA containing a protease inhibitor cocktail. Insoluble cellular components were removed by centrifugation at 15,000 RPM for 10 min at 4° C. The protein content of the supernatant was determined according to the Bradford protein assay using BSA as the standard.

The enzymatic assay was performed using 5 μg protein per sample as described previously. To test the inhibitory effect of AD-3281, at the same conditions, AD-3281 was also added to 0.33 μg recombinant human methionine aminopeptidase 2 obtained from bio-techne (USA, 3795-ZN), samples were incubated with the inhibitor for 15 min at RT before adding the substrate. The reaction was started by adding 250 μM L-Met-AMC as a substrate. An increase of fluorescence, due to substrate degradation during the enzymatic assay, was measured every 20 seconds for 1 h and 30 min at 37° C. using a plate reader (Wallac 1420 VICTOR plate reader, Perkin-Elmer Life Sciences, USA). The assay was carried out on a 96-well plate on ice and it was performed in an assay buffer (pH 7.5) containing 50 mM HEPES, 0.1 mM CoCl2, 100 mM NaCl and 1 mg/mL PEG 6000, in a final volume of 100 μL.

### Cell Growth and Proliferation Assays

A375 were seeded (2000 cells/well) exposed to TNP-470 and to arrange concentrations of AD-3281 (1-100 μM) cells were incubated for 72 h at 37° C., After incubation, MTT (Sigma Aldrich, St. Louis City, MO, USA) was added (0.5 mg/mL) into each well for viability detection and incubated at 37° C. and 5% CO2 for 40 minutes. The absorbance was measured at 540 nm using a plate reader (Wallac 1420 VICTOR plate reader, Perkin-Elmer Life Sciences, USA). The proliferation of HUVECs was measured under the same conditions. HUVECs were seeded (3000 cells/well) in 96-well plates

To further verify the inhibition activity of encapsulated AD-3281 in PLGA nanoparticles HUVECS and A375 were exposed to different concentrations of nanoparticles equivalent to 50-1000 nM free AD-3281 (0.2-4.16 mg/ml nanoparticles) empty nanoparticles were added as a control. The effect of AD-3281 on HUVECs growth was evaluated after 5 days.

### Spheroid Formation Using Multi Well Array

3D Petri Dish 35-well array (Microtissues Inc., RI, USA) was used to create 2% agarose hydrogel micro-wells. Each well contained 5,000 cells of A375 cells. 30 minutes after seeding, 1 ml of medium was added and templates were incubated at 37° C. for 24 hours. Spheroids were treated with free and encapsulated AD-3281 10 μM and 50 μM respectively, after 96 h of incubation spheroids viability was measured using WST1 assay. The absorbance was measured at 450 nm using a plate reader (Wallac 1420 VICTOR plate reader, Perkin-Elmer Life Sciences, USA).

### Animal Models

Animal studies were approved by the Institutional Animal Care and Use Committee of the Faculty of Medicine of the Hebrew University and followed the guidelines for use of laboratory animals. 6-8-week-old male Foxn1 nu mice were S.C. injected with 5×10⁶ A375 and MDA-MB-231 cells/mouse. Mice were I.P treated with 30 mg/kg and 15 mg/kg AD-3281 every other day and with 15 mg/kg, 7.5 mg/kg AD-3281 every other day in mice injected with A375 and MDA-MB-231 respectively. Tumor growth was measured with a digital caliper. At the end of the experiments after 18 or 15 days, mice were sacrificed, and tumors were surgically removed, weighed, volume was measured and histology stating was performed. AD-3281 was dissolved in 10:10:80 chromophore:ethanol:saline.

### Preparation of AD-3281 Loaded Nanoparticle

Nanoparticles were prepared using emulsification evaporation method. AD-3281 was loaded in 100 mg of PLGA 50:50 lactic to glycolic acid ratio. PLGA polymer was dissolved in 10 ml ACN 0.01% Tween 80, AD-3281 was added to the dissolved polymer. Under conditions stirring for 10 minutes using a head stirrer, the organic phase was added to the aqueous Solutol solution. Next, the solution was transferred to a dry round bottom flask and connected to a rotary evaporator. After the solvent was completely evaporated nanoparticles were centrifuged at 10,000 rpm for 10 minutes, re-suspend in 20% trehalose, and lyophilized.

Coumarin-6 as a labeling agent was loaded in PLGA nanoparticles using the same protocol. Empty vehicles were prepared using the same technique and conditions without the addition of AD-3281 or coumarin-6.

### Physiochemical Characterization of Nanoparticles

Nanoparticles' size and charge were measured with dynamic light scattering (DLS) and with Malvern Zetasizer (Malvern Instruments, UK) providing mean size and size range, as ell as charge. All measurements were done at 25° C. nanoparticles were dispersed in d.d.w.

To study the morphology of the nanoparticles, Teansition Electron Microscope (TEM) images were taken. Samples were stained with 2.5% Uranyl acetate. Briefly, 5 μl of samples were placed on formvar/carbon-coated copper 200 mesh grids (EMS), mixed with 5 μl NV (NANOVAN, Nanoprobes, NY, USA) for 5-10 sec, the excess stain was blotted off and grids were dried. Grids were viewed with Jeol® JEM-1400 Plus TEM (Jeol®, Tokyo, Japan), equipped with ORIUS SC600 CCD camera (Gatan®, Abingdon, United Kingdom), and Gatan Microscopy Suite program (DigitalMicrograph, Gatan®, UK).

### Uptake of Cancer Cells

To evaluate the uptake of the PLGA nanoparticles by A375, 6-coumarin was encapsulated into the PLGA polymers using as a labeling agent. A375 were seeded in a 6-well plate 300,000 cells/well. After 24 h fluorescent nanoparticles (10 mg/ml) were suspended using a bath sonicator for 5 min and 75 μl were added to the seeded cells. After 0, 4, 7, and 24 hours. After the incubation with particles, cells were washed three times with cold phosphate-buffered saline (PBS) followed by a detachment using trypsin, washed again, and fixed by 4% paraformaldehyde, and analyzed by FACS (BD LSRFortessa™)

### Statistical Analysis

Results are presented as mean±SEM. Studies containing more than three groups were compared and analyzed using a one-way analysis of variance (ANOVA) and significant differences were detected using Tuckey's multiple comparison post-test. Differences were considered statistically significant for p<0.05.

### Results

### Synthesis and Characterization of AD-3281

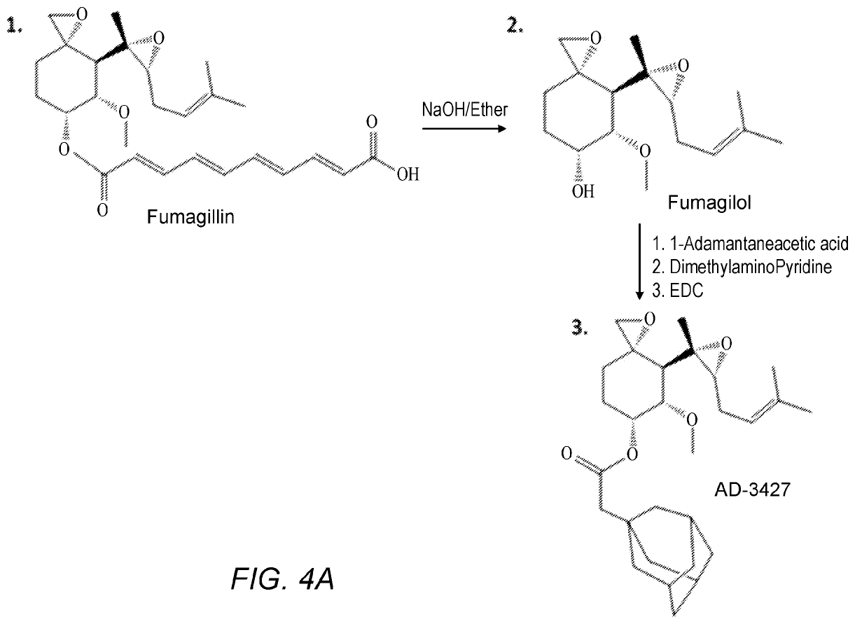

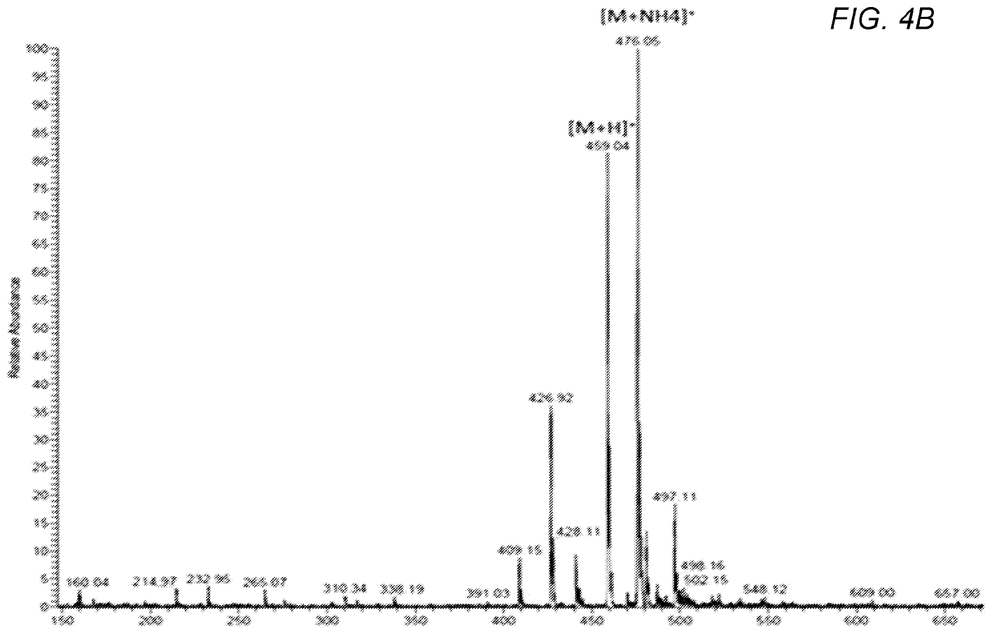

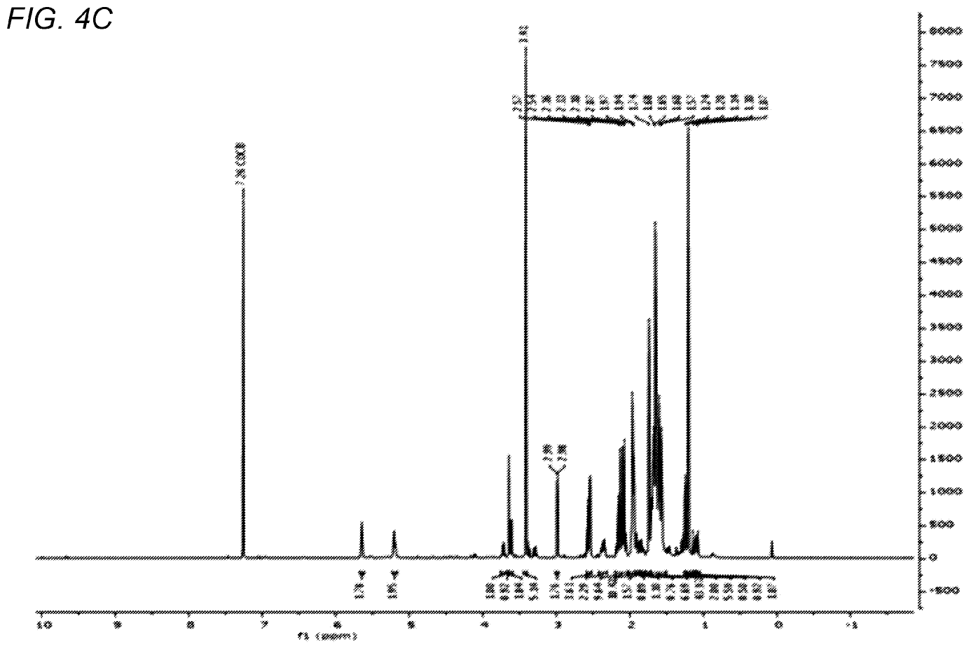

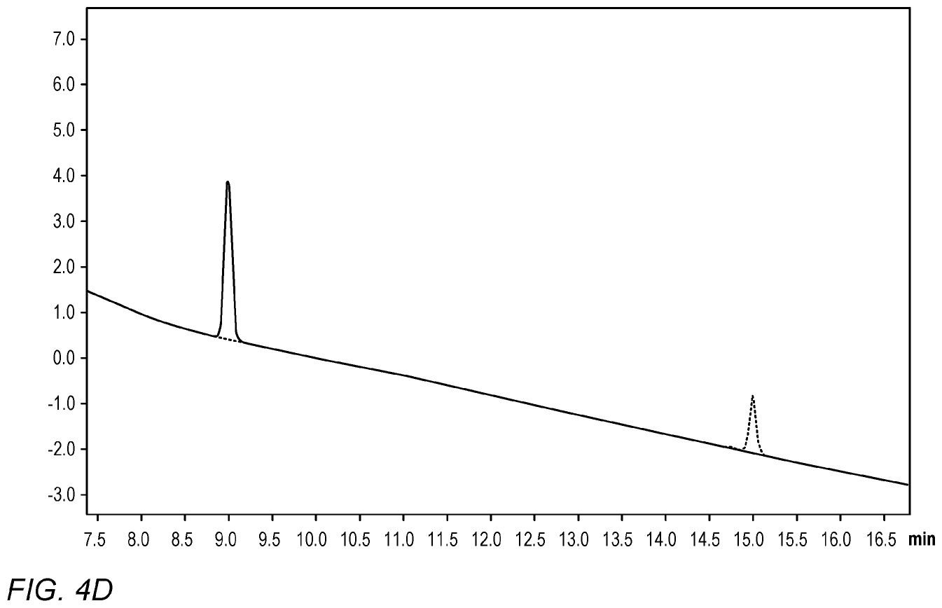

Fifteen fumagillol derivatives with fumagillol backbone and varying side groups were synthesized and tested. Among the various analogs, AD-3281 represents the most potent compound. AD-3281 synthesis started with fumagillin and under basic conditions alkaline hydrolysis of fumagillin yields fumagillol. The synthesis then was completed by esterification reaction with 1-adamantane-acetic acid (FIG. 4A) illustrates the synthesis of AD-3281. The chemical structure of the new inhibitor was confirmed by mass spectrometry and H NMR (FIGS. 4B,C). The retention time of AD-3281 was determined by high-performance liquid chromatography (HPLC) (FIG. 4D).

### MetAp2 Expression and Inhibition

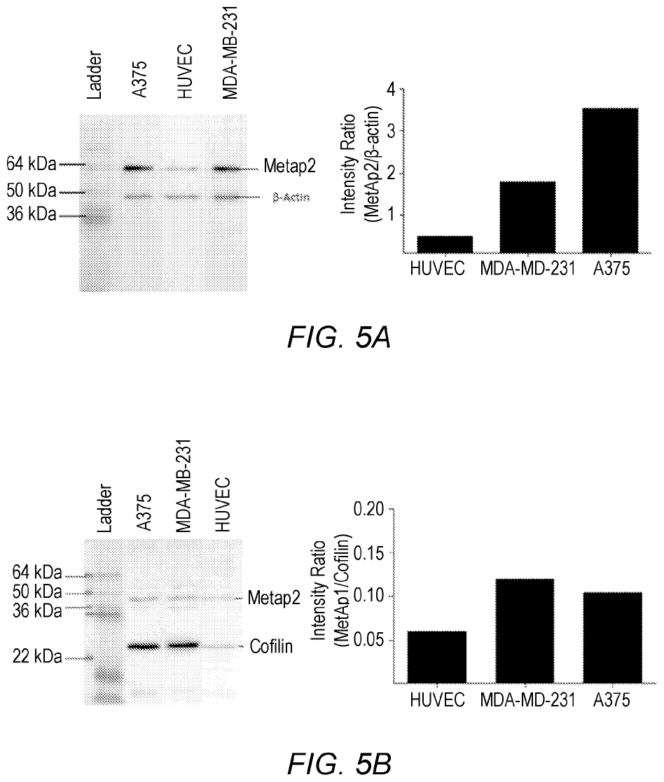

The basal cellular protein expression of MetAp2 was determined using western blot. Compared with endothelial cells, cancer cells showed a higher expression of MetAp2. In A375 cancer cells, a higher level of MetAp2 was observed when compared with MDA-MB-231. (FIG. 5)

In order to study the inhibition of MetAp2 enzyme activity in response to AD-3281, the compound was added to human recombinant MetAp2, fumagillol analog, TNP-470 was used as a positive control. The N-terminal methionine excision activity was compared. We found that 2.5 μM of AD-3281 led to a 95% reduction in MetAp2 activity compared with the untreated control.

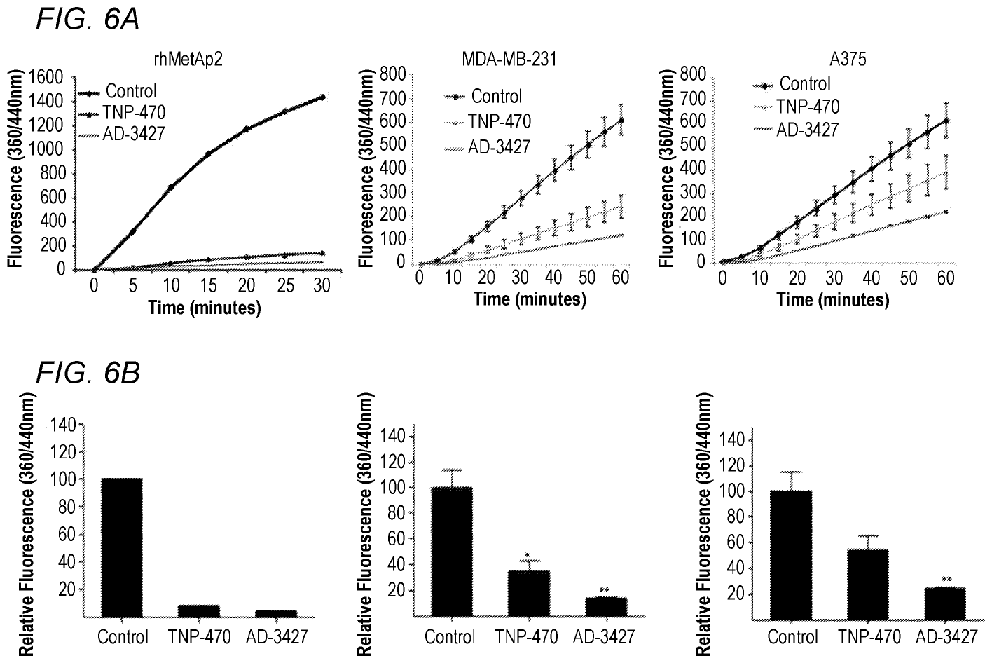

MetAp2 activity in A375 and MDA-MB-231 was also measured under the same conditions after the treatment of 2.5 μM of AD-3281. In this assay, we found that AD-3281 reduced the enzymatic activity in both cell lines by 64% and 80% respectively over the course of 1 h (FIG. 6A). Significant inhibition was obtained after 10 minutes of AD-3281 exposure in both cancer cell lines (FIG. 6B).

### AD-3281 Affect the Proliferation of Endothelial and Cancer Cells

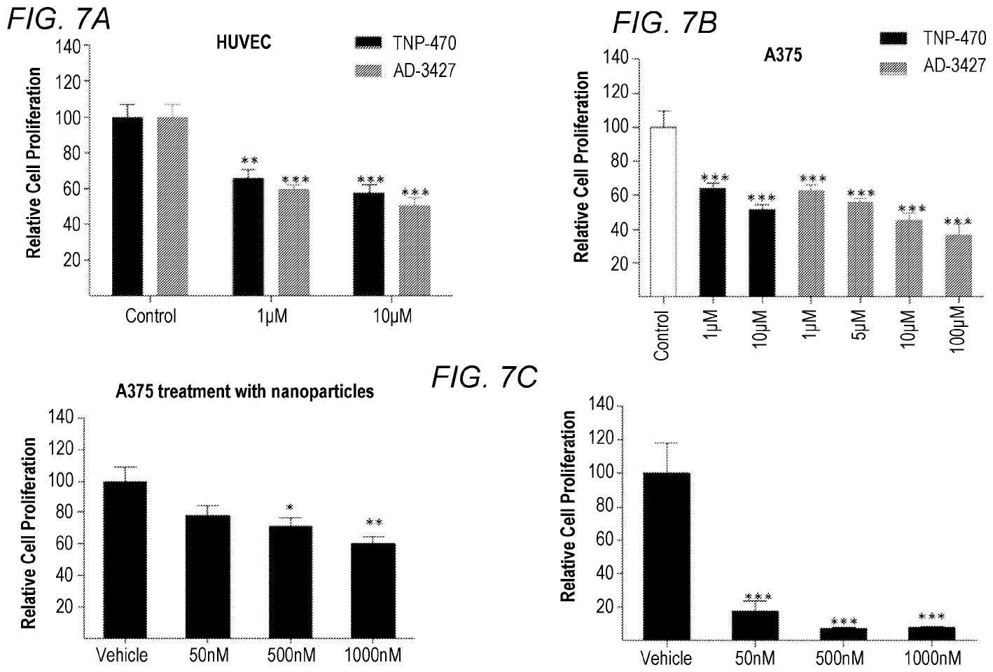

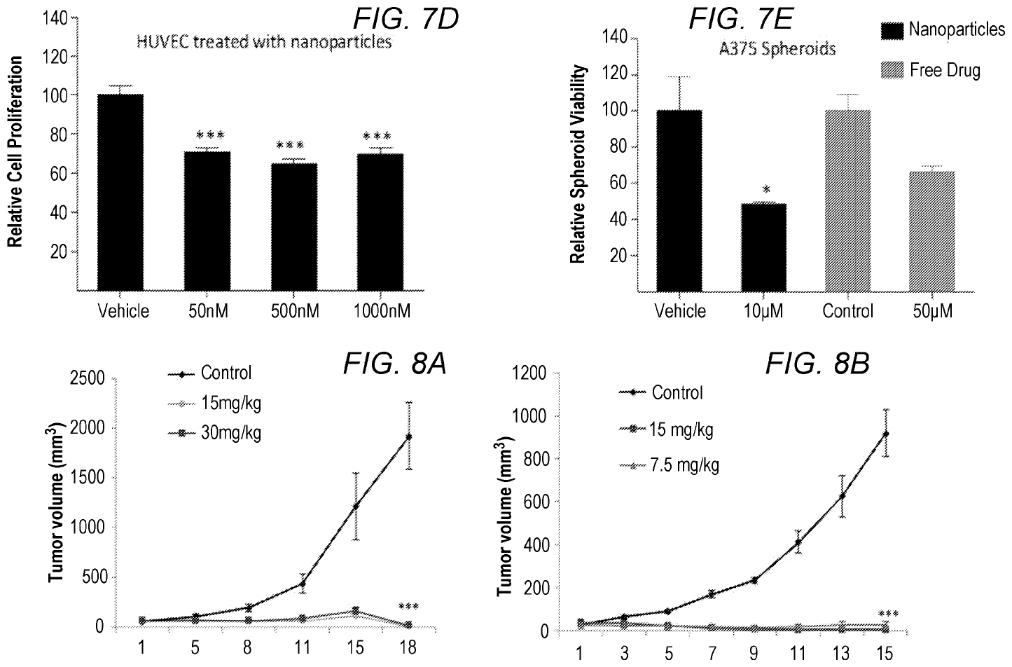

To study the anti-angiogenic activity of AD-3281, MTT viability assay was conducted to assess the inhibition effect of on endothelial cell proliferation. HUVEC cells were seeded and treated with 1 μM and 10 μM of AD-3281. After 72 h of incubation, AD-3281 showed a significant reduction in HUVECs proliferation by 40 and 50% respectively. (FIG. 7A)

To further examine the inhibition activity of AD-3281 on cancer cell proliferation, an MTT assay was also conducted on A375 cancer cells. Cells were exposed to different concentrations of AD-3281 (1-100 μM) which resulted in a significant dose-dependent reduction in cancer cell proliferation by 37-63% respectively. (FIG. 7B)

In order to increase the cellular bioavailability and to facilitate the dissolution of the hydrophobic new compound, we formulated AD-3281 into PLGA nanoparticles using the emulsification evaporation method. We assessed the effect of the encapsulated AD-328ion the proliferation of endothelial cells. HUVECs were treated q.o.d with PLGA-AD-3281 (500 nM-1000 nM AD-3281 equivalent) and the growth of HUVECs was evaluated using MTT assay after 5 days. Treated cells showed ˜31% inhibition in cell proliferation compared with cells exposed to vehicle only. Similarly, the effect of AD-3281 nano formulation on the proliferation of A375 cancer cells was tested. After 72 h a dose-dependent inhibition was observed in treated cells with the encapsulated AD-3281 (500 mM-1000 mM AD-3281 equivalent) (22-40%). The incubation period of 96 h showed an effective reduction in the growth of A375 cells by 82-92%. (FIGS. 7C,D). The interaction of the nanoparticles with cells were investigated using uptake assays confirming the uptake of polymeric nanoparticles by A375 with a level of ˜20% after 7 hours of incubation.

The inhibition activity of AD-3281 on A375 cells was evaluated in 3d multicellular spheroids that exhibit spatial cell-cell interaction, proliferation, and show gradient of nutrients due to diffusion of drugs similarly to in vivo. Therefore 3D spheroid are expected to be a better predictive to the performance in vivo. Spheroid viability was measured using WST1 assay; spheroids were exposed to 10 μM equivalent to AD-3281 and to and 50 μM free inhibitor. After 96 h, spheroids treated with encapsulated inhibitor induced a reduction of 51% in spheroids viability compared to spheroids treated with vehicle only. Spheroids treated with the free inhibitor did not show a significant reduction of the spheroid's viability. (FIG. 7E)

### AD-3281 Inhibits Tumor Growth

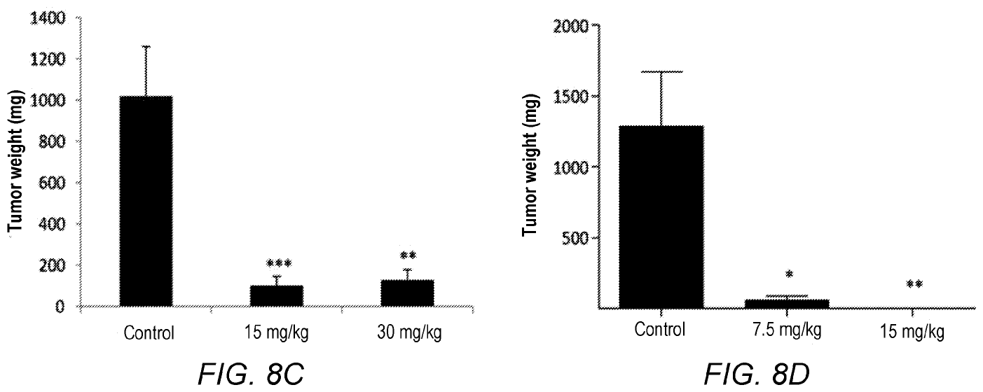

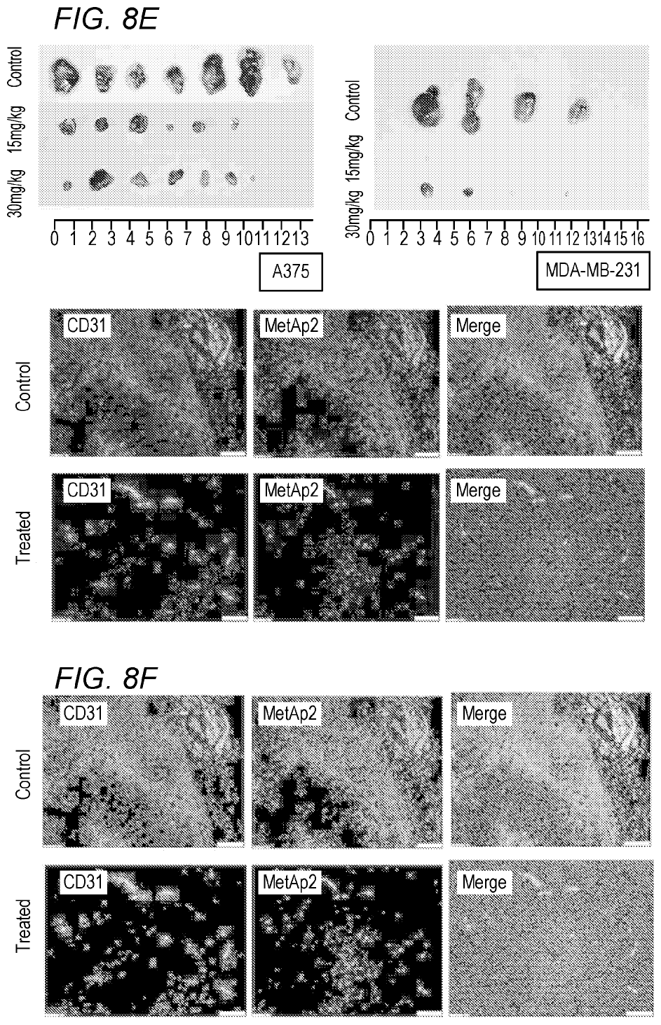

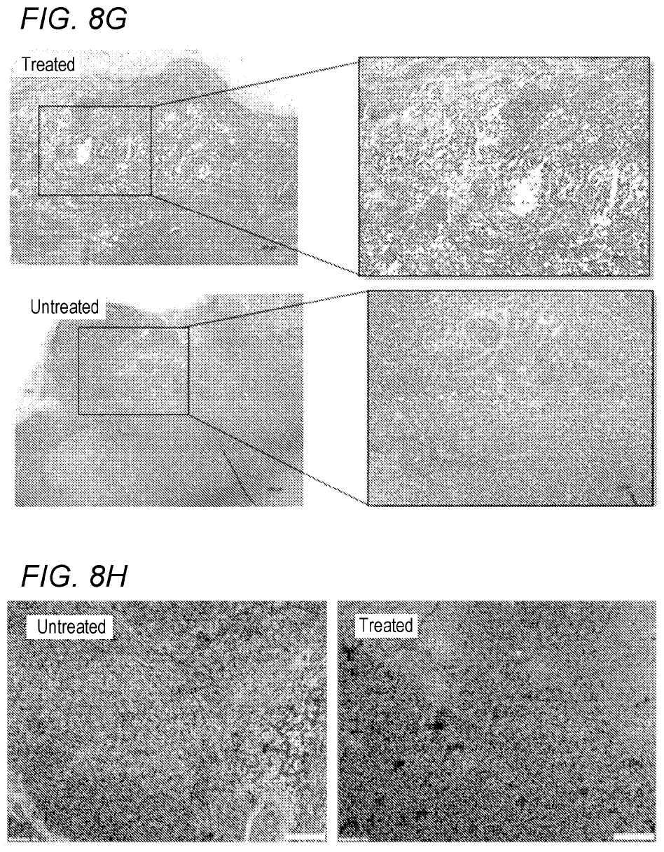

In vivo studies were performed in two cancer xenograft models. Mice were injected subcutaneous (s.c) with 5×10⁶ A375 cancer cells, different intraperitoneal (I.P.) dosing of AD-3281 was initiated when tumors reach a size of ˜100 mm³, 15 mg/kg, and 30 mg/kg q.o.d. After 8 days AD-3281 resulted in a significant inhibition of ˜70% in tumor growth in treated groups when compared with the control, and at day 18 tumor growth was inhibited by 99%. (FIG. 8A). At the endpoint, on day 18 the mean weight of extracted tumors from the untreated group was 1 g and the treated groups had a mean weight of ˜0.1 g (FIG. 8C). The immunofluorescent staining of sections obtained from the extracted tumors revealed that MetAp2 and CD31 were co-localized and expressed in both the treated and untreated tumors. However, in the treated samples, co-localization appeared to a lesser extent and complete blood vessels were observed more clearly in untreated groups. (FIGS. 8F,G). Furthermore, AD-3281-treated tumors showed less cellular proliferation than untreated tumors this was detected by the nuclear marker Ki-67. (FIG. 8H).

In s.c. MDA-MB-231 xenograft study, tumors were also inhibited when treated with 7.5 mg/kg and 15 mg/kg q.o.d with AD-3281. The treatments were effective already after 5 days and after 15 days 97% volume inhibition was obtained in the treated group with 7.5 mg/kg whereas 3 tumors seemed to be completely eradicated in the group treated with 15 mg/kg. (FIG. 8B) Extracted tumors had a mean weight of 1 g in the untreated group in treated groups with 7.5 mg/kg and 15 mg/kg the mean weight was 0.05 and 0.001 g respectively. (FIG. 8D). Immunohistological structure of MDA-MB-231 tumor-bearing mice showing similar trends as in melanoma with reduction in vascularization.

### Discussion

Tumor growth and metastasis depend on the angiogenesis process, whereas the inhibition of angiogenesis was established as an important modality for tumor suppression and spread when combined with chemotherapeutic drugs. While there are range of inhibitors that reached clinical approval, many of them are not sufficiently efficient or carry various side effects. Therefore, finding new angiogenic inhibitors, with high potency and drug-like properties may open up new avenue in cancer treatment, especially given the lower toxicity profile of these agents compared with chemotherapies.

Many studies established that MetAP2 plays an important role in the development of various types of cancer and the specific downregulation of human MetAP2 expression by an antisense oligonucleotide were predominantly affect endothelial cell proliferation. In our recently paper, we found the involvement of MetAP2 in lymphangiogenesis, indicating a dual action of MetAp2 in both vascular and lymphatic capillary formation. Therefore, there is a rationale for positioning MetAp2 as a useful target for the treatment of primary cancers as well as metastatic disease.

One of the most effective known inhibitors of MetAp2 is originated from the natural compound Fumagillin. This small molecule was isolated from Aspergillus fumigatus Fresenius, and the synthetic analogue, O-(chloroacetylcarbamoyl) fumagillol or TNP-470, (also referred to as AGM-1470) was one of the most potent analogs of fumagillin as demonstrated in angiogenesis cell models and one of the first anti-angiogenic small molecule drugs to undergo clinical trials. However, the development of this derivate was hindered by major clinical drawbacks related to dose-depending side effects. The high potential of fumagillol derivates motivated us and others to search for new safe compounds with high activity.

The selected lead compound, AD-3281 was the most active in suppressing endothelial cell proliferation and arrest MeAp2 enzymatic activity.

It is well established that MetAp2 is overexpressed in the tumor microenvironment, and most significantly in the endothelium. We found that MetAp2 was also highly expressed in cancer cells in comparable level to endothelial cells and in the enzymatic assay AD-3281 markedly suppressed the proteolytic activity of MeAp2 (FIG. 6). It was indicated that the inhibition of MetAp2 enzymatic activity leads to angiogenesis blockage and suppress tumor growth.

Furthermore, we aimed to investigate the effect of AD-3281 on cellular functionality in cancer and endothelial cells. MetAp2 inhibition in vascular endothelium is known to regulate cell proliferation through cell cycle arrest in the late G₁ phase. We found that the presence of AD-3281 in human melanoma cell line, A375, as well as the primary endothelial cells, HUVEC, impaired cell proliferation in range of concentration of 1-100 μM. The relatively high concentration required for suppression was attributed to the low solubility in water. In many cased shifting to particulate carriers can trigger cell uptake via endocytosis thus improve access of drugs to cells. Therefore to increase cellular availability we devised a nano-formulation based on PLGA biodegradable polymer at the mean size of 194.0±88.06 nm. Our previous studies showed that capacity to enhance bioavailability of lipophilic drugs, as well as improve their retention and stability in vivo, using 10-100 folds less of a dose than the free drug. Similarly, the inhibition effect of the encapsulated AD-3281 on the proliferation of endothelial and A375 cells showed a significant improvement of bioavailability of AD-3281 with almost 20 fold reduction in dose compared with the free molecule (FIG. 7). Unlike 2D cell cultures, in 3D cell assembly and interact spatially thus showing more physiologically relevance and provide better prediction of drug efficacy compared to monolayer cell cultures. To generate a robust spheroid growth, we use a lab-designed 3D-printed multi-well array that can generate uniform 3D cultures with high reproducibility. In the spheroid model, encapsulated AD-3281 was found to significantly suppress spheroid growth, while the free AD-3281 showed a slight reduction of spheroids viability. This can be explained by the improvement of the penetration of AD-3281 in their particulate PLGA nano carrier form, that enables high capacity of trans and intra cellular transport.

The in-vitro observations correlate with our in vivo results, AD-3281 showed substantial anti-tumor effects and it was observed in two tumor-bearing mice models. This shows the broad biological effect of AD-3281. Histological analyses showed that in mice treated with AD-3281 endothelial cells re-modulation was affected. Immunofluorescence of extracted murine tumors tissue sections showed that in treated tissues blood vessels positive cells were organized more sporadically and less collectively as vessels, compared with untreated groups. Moreover, AD-3281 reduced the cellular proliferation in treated tumors; this was detected by the nuclear marker ki-67.

Taken together, our data shows that our new inhibitor AD-3281 has promising therapeutic properties in the treatment of cancer progression; this novel inhibitor demonstrated an effective inhibition role in blood vascularization and tumor progression in tumor-bearing mice. These significant results were mainly attributed to its anti-angiogenic and anti-cancer activity. Our outcomes using AD-3281 emphasize it as a great potential compound for treating highly vascularized tumors it may be useful for cancer patients as a long-term maintenance drug to prevent tumor recurrence.

### Experimental Set-Up 2

### Cell Culture

Human umbilical vein endothelial cells were purchased from Lonza (Walkersville, MD, USA) and were grown in PeproGrow endothelial cell media supplemented with MacroV with 1% penicillin/streptomycin. MDA-MB-231 and A375 were purchased from ATCC and were maintained in Dulbecco's modified eagle's medium (DMEM), supplemented with 10% fetal calf serum (FCS) media with 1% penicillin/streptomycin. Cells kept in humidified incubated at 37° C. with 5% CO₂. All cells were characterized before use, mycoplasma-free, using an EZ-PCR Mycoplasma Test Kit (Biological Industries).

### Western Blot

Cells extracts were isolated using RIPA buffer with protease inhibitor cocktail (Sigma, S8820) for 30 min on ice. Lysates were centrifuged, and the supernatant was collected. Using BCA Protein Assay kit (Pierce™, Thermo Fisher Scientific, Cambridge, MA, USA) isolated protein content was determined. Proteins (15 μg protein) were separated by a 12.5% Tris-glycine SDS-PAGE and transferred onto a Polyvinylidene difluoride membrane (Millipore Corporation, Billerica, MA, USA). The Membranes were incubated in blocking buffer for 2 h and then incubated with anti-MetAp2 abs or anti-MetAp1 abs, Ab134124 or Ab185540 (Abcam, Cambridge, UK), respectively, overnight at 4° C. in TBST containing 5% BSA. After washing three times for 5 min in TBST, the membranes were incubated with a 1:5000 dilution of goat anti-rabbit secondary ab conjugated to horseradish peroxidase for 1 h (Ab97080, Abcam). 0-actin or cofilin, Ab49900 or Ab124979 (Abcam), respectively, were used as the loading control.

### MetAp2 Enzymatic Activity Assays

A375 and MDA-MB-231 were sub-cultivated using trypsin, then centrifuged and counted. To obtain a homogenate containing an equivalent cell number, cells were resuspended in an appropriate volume of cold RIPA containing a protease inhibitor cocktail. Insoluble cellular components were removed by centrifugation at 15,000 RPM for 10 min at 4° C. The protein content of the supernatant was determined according to the Bradford protein assay using BSA as the standard. The enzymatic assay was performed using 5 μg protein per sample as described previously. To test the inhibitory effect of AD-3281, at the same conditions, AD-3281 was also added to 0.33 μg recombinant human methionine aminopeptidase 2 obtained from bio-techne (USA, 3795-ZN), samples were incubated with the inhibitor for 15 min at RT before adding the substrate. The reaction was started by adding 250 μM L-Met-AMC as a substrate. An increase of fluorescence, due to substrate degradation during the enzymatic assay, was measured every 20 seconds for 1 h and 30 min at 37° C. using a plate reader (Wallac 1420 VICTOR plate reader, Perkin-Elmer Life Sciences, USA). The assay was carried out on a 96-well plate on ice and it was performed in an assay buffer (pH 7.5) containing 50 mM HEPES, 0.1 mM CoCl₂, 100 mM NaCl and 1 mg/mL PEG 6000, in a final volume of 100 μL.

### Cell Growth and Proliferation Assays

A375 were seeded (2000 cells/well) exposed to TNP-470 and to arrange concentrations of AD-3281 (1-100 μM) cells were incubated for 72 h at 37° C., after incubation, MTT (Sigma Aldrich, St. Louis City, MO, USA) was added (0.5 mg/mL) into each well for viability detection and incubated at 37° C. and 5% CO₂ for 40 minutes. The absorbance was measured at 540 nm using a plate reader (Wallac 1420 VICTOR plate reader, Perkin-Elmer Life Sciences, USA). The proliferation of HUVECs was measured under the same conditions. HUVECs were seeded (3000 cells/well) in 96-well plates.

To further verify the inhibition activity of encapsulated AD-3281 in PLGA nanoparticles HUVECs and A375 were exposed to different concentrations of nanoparticles equivalent to 50-1000 nM free AD-3281 (0.2-4.16 mg/ml nanoparticles) empty nanoparticles were added as a control. The effect of AD-3281 on HUVECs growth was evaluated after 5 days.

### Tube Formation Assay

To evaluate the angiogenic effect of AD-3281, an endothelial tube formation assay was performed. HUVECs were kept in serum-free Dulbecco's modified Eagle's medium media, collected, seeded in 0.1% gelatin coated 96-well plate and monitored for 12 h using a microscope. Images were taken from 6 separated wells and analyzed using Incucyte® Live-Cell Analysis Systems.

### Spheroid Formation Using Multi Well Array

3D Petri Dish 35-well array (Microtissues Inc., RI, USA) was used to create 2% agarose hydrogel micro-wells. Each well contained 5,000 cells of A375 cells. 30 minutes after seeding, 1 ml of medium was added and templates were incubated at 37° C. for 24 hours. Spheroids were treated with free and encapsulated AD-3281 10 μM and 50 μM respectively, after 96 h of incubation spheroids viability was measured using WST1 assay. The absorbance was measured at 450 nm using a plate reader (Wallac 1420 VICTOR plate reader, Perkin-Elmer Life Sciences, USA).

### Animal Models

Animal studies were approved by the Institutional Animal Care and Use Committee of the Faculty of Medicine of the Hebrew University and followed the guidelines for use of laboratory animals. 6-8-week-old male Foxn1 nu mice were S.C. injected with 5×10⁶ A375 or MDA-MB-231 cells/mouse. Mice were I.P treated with 30 mg/kg and 15 mg/kg AD-3281 q.o.d and with 15 mg/kg, 7.5 mg/kg AD-3281 (dissolved in 10:10:80 Cremophor EL: Ethanol: Saline) q.o.d in mice injected with A375 and MDA-MB-231 respectively. Tumor growth was measured with a digital caliper. At the end of the experiments after 18 or 15 days, mice were sacrificed, and tumors were surgically removed, weighed, volume was measured and histology stating was performed. Tumor tissues were resected at the end point and analyzed by immunohistology using standard protocol for paraffin fixed sections using anti-Ki-67 (Abcam, catalog number ab15580), anti-CD31 (Abcam, catalog number ab28364), anti-MetAp-2 (Ab134124, Abcam, Cambridge, UK) (Figure S.

### Preparation of AD-3281 Loaded Nanoparticle

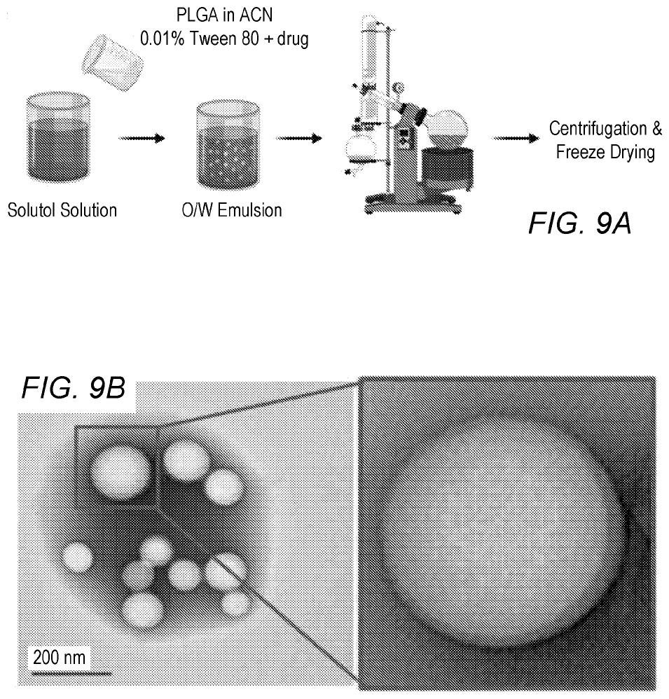

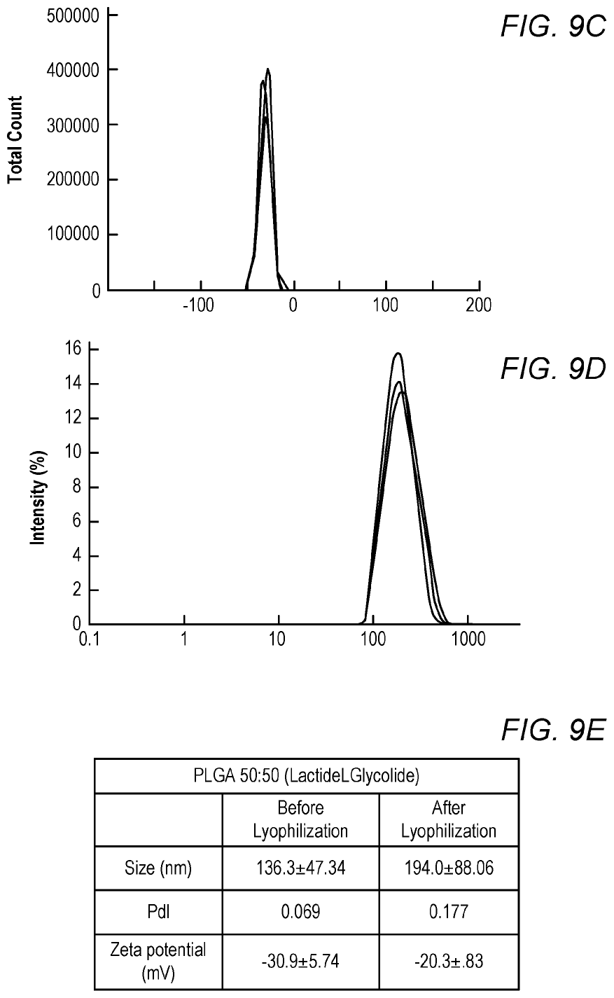

Nanoparticles were prepared using emulsification evaporation method. AD-3281 was loaded in 100 mg of PLGA 50:50 lactic to glycolic acid ratio (50:50, acid terminated, Sigma-Aldrich, cat: 719900). PLGA polymer was dissolved in 10 ml ACN 0.01% Tween 80, AD-3281 was added to the dissolved polymer under stirring for 10 minutes using an overhead stirrer, the organic phase was added to the aqueous Solutol solution. Next, the solution was transferred to a round bottom flask and connected to a rotary evaporator. After the solvent was completely evaporated nanoparticles were centrifuged at 10,000 rpm for 10 minutes, re-suspend in 20% trehalose, and lyophilized. (Steps are shown in FIG. 9a). Coumarin-6 (0.1% w/w) was used to fluorescently label the PLGA nanoparticles using the same protocol. Empty vehicles were prepared using the same technique and conditions without the addition of loaded compound.

### Physiochemical Characterization of Nanoparticles

Nanoparticles' size and charge were measured with dynamic light scattering (DLS) and with Malvern Zetasizer (Malvern Instruments, UK) providing mean size and size range, as well as charge. All measurements were done at 25° C. nanoparticles were dispersed in d.d.w.

To study the morphology of the nanoparticles, Transition Electron Microscope (TEM) images were taken. Samples were stained with 2.5% Uranyl acetate. Briefly, 5 μl of samples were placed on formvar/carbon-coated copper 200 mesh grids (EMS), mixed with 5 μl NV (NANOVAN, Nanoprobes, NY, USA) for 5-10 see, the excess stain was blotted off and grids were dried. Grids were viewed with Jeol® JEM-1400 Plus TEM (Jeol®, Tokyo, Japan), equipped with ORIUS SC600 CCD camera (Gatan®, Abingdon, United Kingdom), and Gatan Microscopy Suite program (DigitalMicrograph, Gatan®, UK).

### Uptake of Cancer Cells

To evaluate the uptake of the PLGA nanoparticles by A375, 6-coumarin was encapsulated into the PLGA polymers using as a labeling agent. A375 were seeded in a 6-well plate 300,000 cells/well. After 24 h fluorescent nanoparticles (10 mg/ml) were suspended using a bath sonicator for 5 min and 75 μl were added to the seeded cells. After 0, 4, 7, and 24 hours. After the incubation with particles, cells were washed three times with cold phosphate-buffered saline (PBS) followed by a detachment using trypsin, washed again, and fixed by 4% paraformaldehyde, and analyzed by FACS (BD LSRFortessa™)

### Statistical Analysis

Results are presented as mean±SEM. Studies containing more than three groups were compared and analyzed using a one-way analysis of variance (ANOVA) and significant differences were detected using Tuckey's multiple comparison post-test. Differences were considered statistically significant for p<0.05.

### Results

### MetAp2 Expression and Inhibition

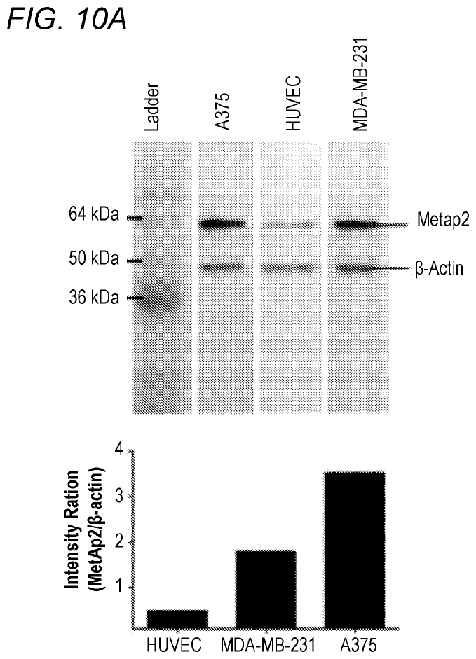

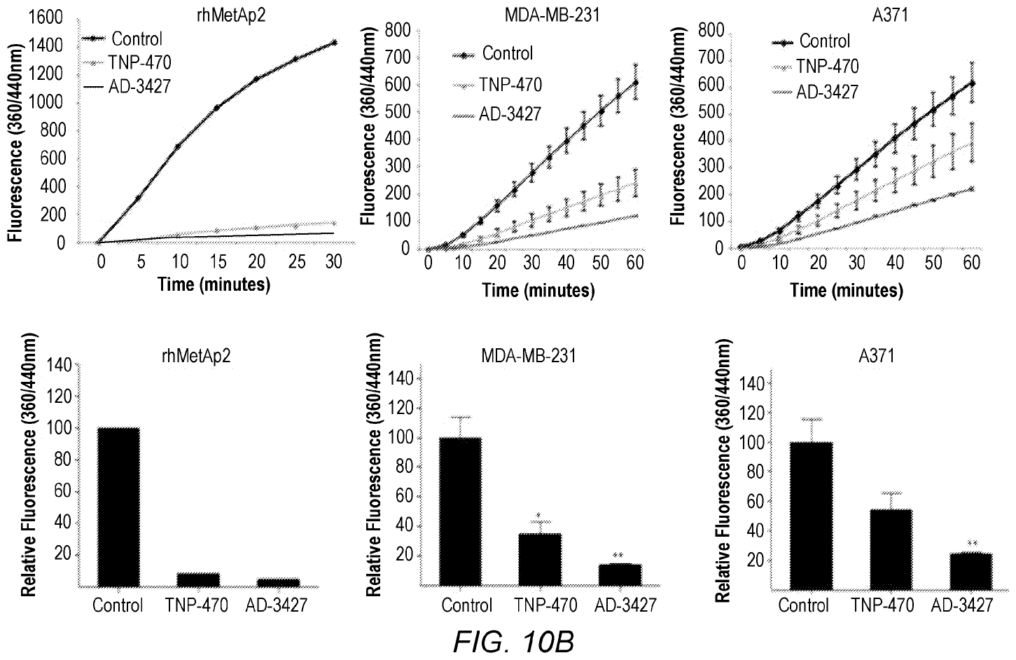

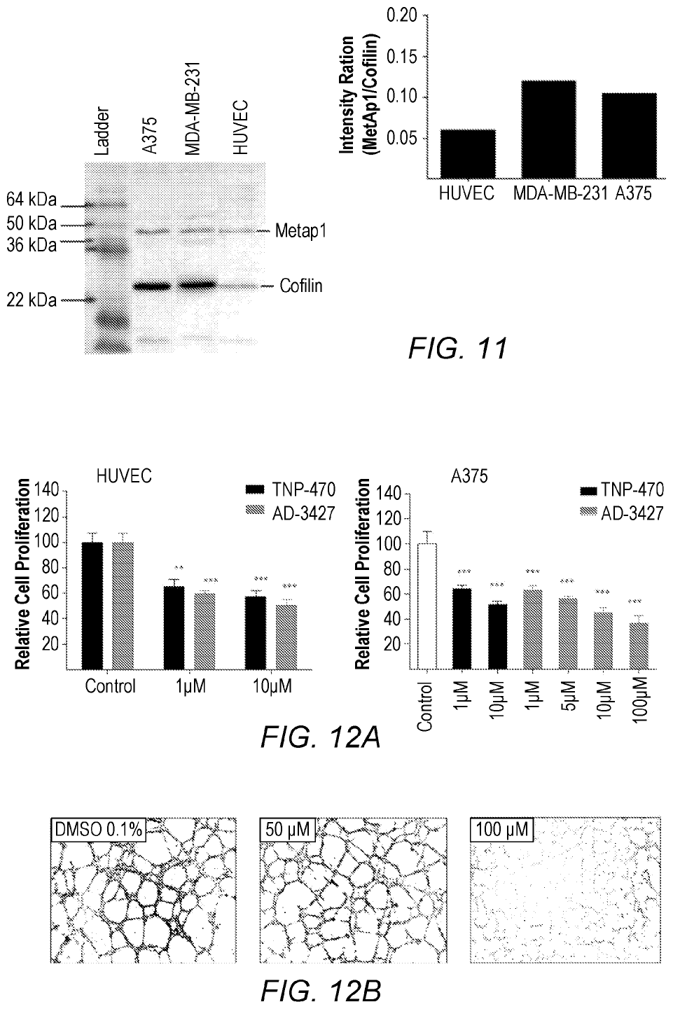

The basal cellular protein expression of MetAp2 was determined using western blot. Compared with endothelial cells, cancer cells showed a higher expression of MetAp2. In A375 cancer cells, a higher level of MetAp2 was observed when compared with MDA-MB-231 (FIG. 10a). The level of MetAp1 was found to be similar in both cancer cell lines and slightly above the level of the expression in HUVEC cells (FIG. 11).

In order to study the inhibition of MetAp2 enzyme activity in response to AD-3281, the compound was added to human recombinant MetAp2, fumagillol analog, TNP-470 was used as a positive control. The N-terminal methionine excision activity was compared. We found that 2.5 μM of AD-3281 led to a 95% reduction in MetAp2 activity compared with the untreated control (FIG. 10b).

MetAp2 activity in A375 and MDA-MB-231 was also measured under the same conditions after the treatment of 2.5 μM of AD-3281. In this assay, we found that AD-3281 reduced the enzymatic activity in both cell lines by 64% and 80% respectively over the course of 1 h (FIG. 10b). Significant inhibition was obtained after 10 minutes of AD-3281 exposure in both cancer cell lines.

### AD-3281 Affect the Proliferation of Endothelial and Cancer Cells and Disrupt Tube Formation

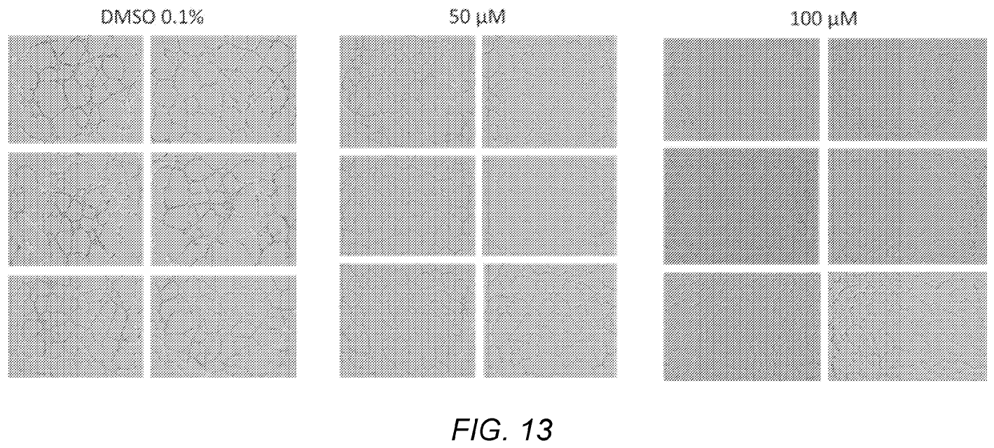

To study the anti-angiogenic activity of AD-3281, MTT viability assay was conducted to assess the inhibition effect on endothelial cell proliferation. HUVEC cells were seeded and treated with 1 μM and 10 μM of AD-3281. After 72 h of incubation, AD-3281 showed a significant reduction in HUVECs proliferation by 40 and 50% respectively (FIG. 12a). To further examine the inhibition activity of AD-3281 on cancer cell proliferation, an MTT assay was also conducted on A375 cancer cells. Cells were exposed to different concentrations of AD-3281 (1-100 μM) which resulted in a significant dose-dependent reduction in cancer cell proliferation by 37-63% respectively. (FIG. 11a). The anti-angiogenic activity of AD-3281 was assessed using the tube formation assay using HUVECs. A concentration of 100 μM AD-3281 was shown to prevent tube formation, while 50 μM seemed to form narrower tubes compared with untreated control but without preventing network formation (FIG. 12b, FIG. 13).

### AD-3281 Inhibits Tumor Growth

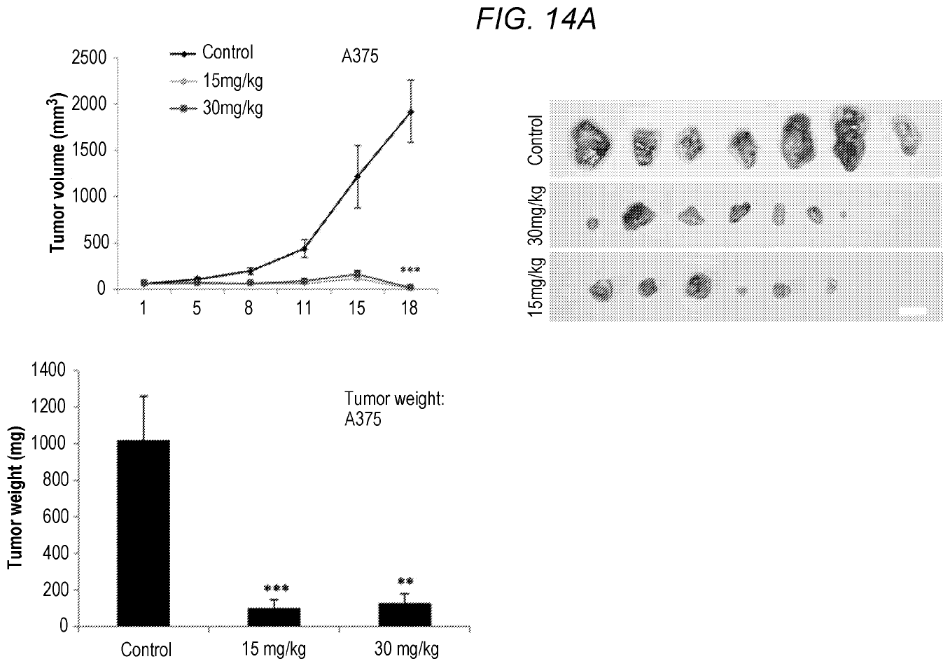

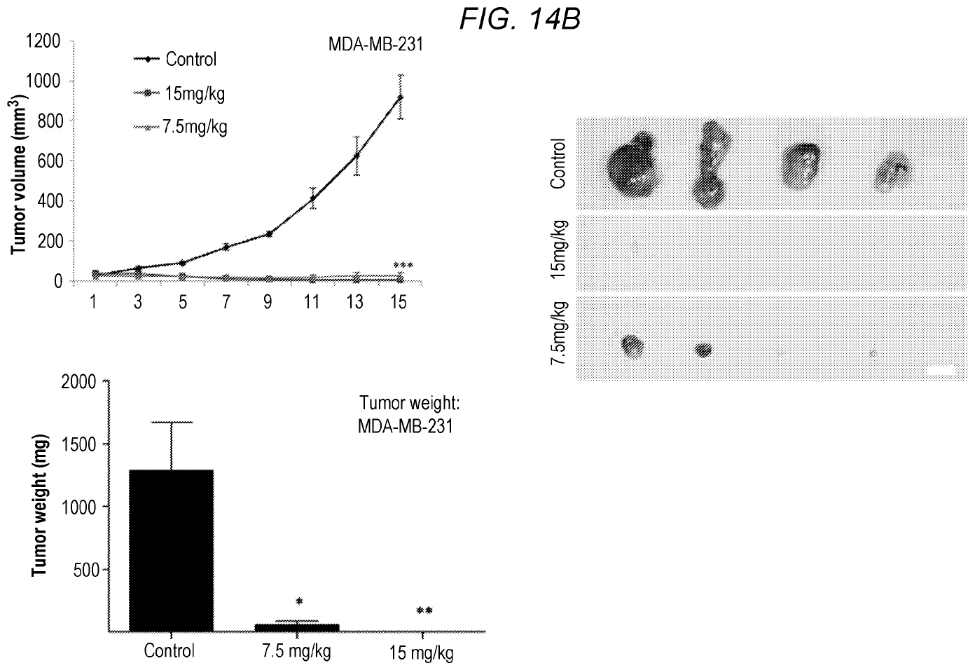

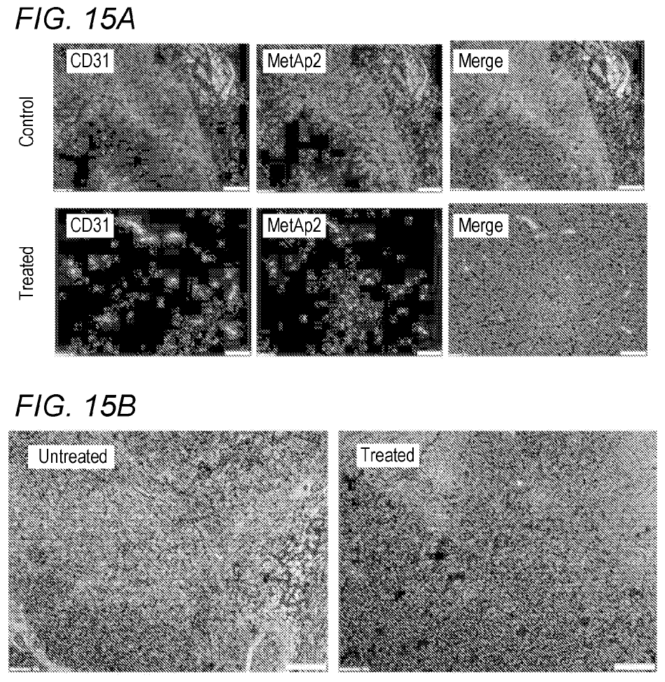

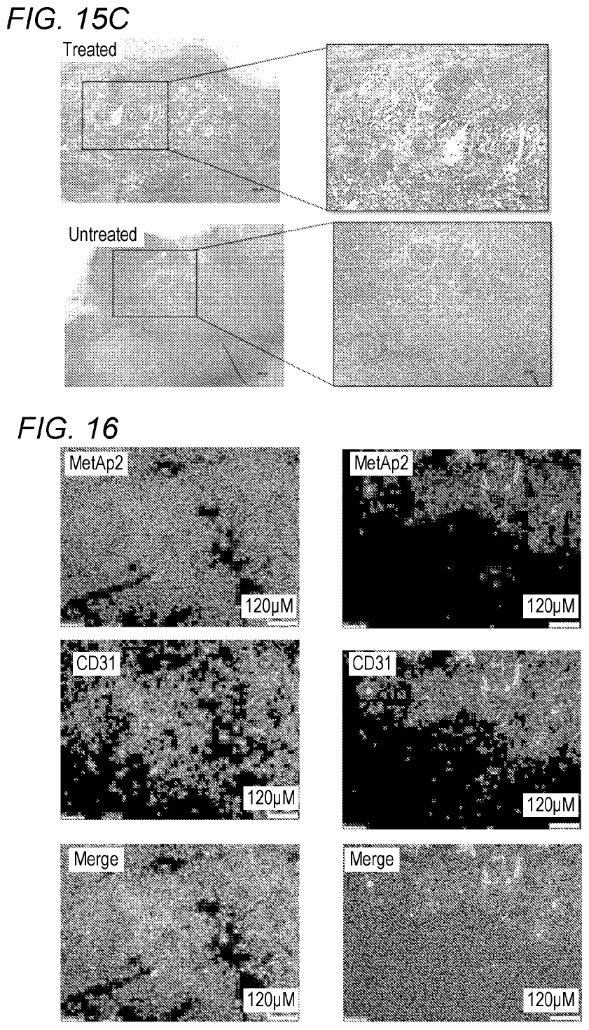

In vivo studies were performed in two cancer xenograft models. Mice were injected subcutaneous (s.c) with 5×10⁶ A375 cancer cells, different intraperitoneal (I.P.) dosing of AD-3281 was initiated when tumors reach a size of ˜100 mm³, 15 mg/kg, and 30 mg/kg q.o.d. After 8 days AD-3281 resulted in a significant inhibition of ˜70% in tumor growth in treated groups when compared with the control, and at day 18 tumor growth was completely inhibited (by 99%) (FIG. 14). At the endpoint, on day 18 the mean weight of extracted tumors from the untreated group was 1 g and the treated groups had a mean weight of ˜0.1 g (FIG. 14a). The immunofluorescent staining of sections obtained from the extracted tumors revealed that MetAp2 and CD31 were co-localized and expressed in both the treated and untreated tumors. However, in the treated samples, co-localization appeared to a lesser extent and complete blood vessels were observed more clearly in untreated groups. (FIG. 15). Furthermore, AD-3281-treated tumors showed less cellular proliferation than untreated tumors this was detected by the nuclear marker Ki-67. (FIG. 15).

In s.c. MDA-MB-231 xenograft study, tumors were also inhibited when treated with 7.5 mg/kg and 15 mg/kg q.o.d with AD-3281. The treatments were effective already after 5 days and after 15 days 97% volume inhibition was obtained in the treated group with 7.5 mg/kg whereas 3 tumors seemed to be completely eradicated in the group treated with 15 mg/kg. (FIG. 14b) Extracted tumors had a mean weight of 1 g in the untreated group. In treated groups with 7.5 mg/kg and 15 mg/kg the mean weight was 0.05 and 0.001 g respectively. Immunobiological structure of MDA-MB-231 tumor-bearing mice showing similar trends as in melanoma with reduction in vascularization. (FIG. 16). All treated mice had no signs of systemic adverse effects and not weight loss was detected (>10%).

### Encapsulation of AD-3281 in PLGA Nanoparticles

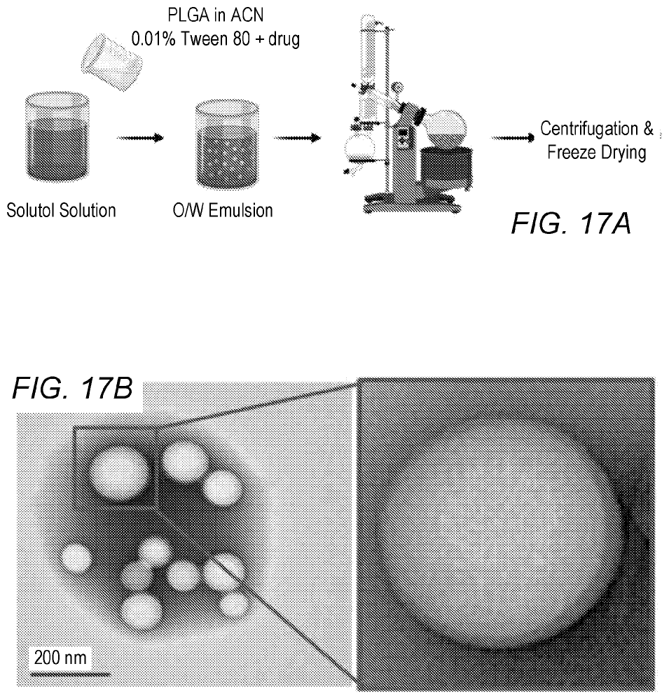

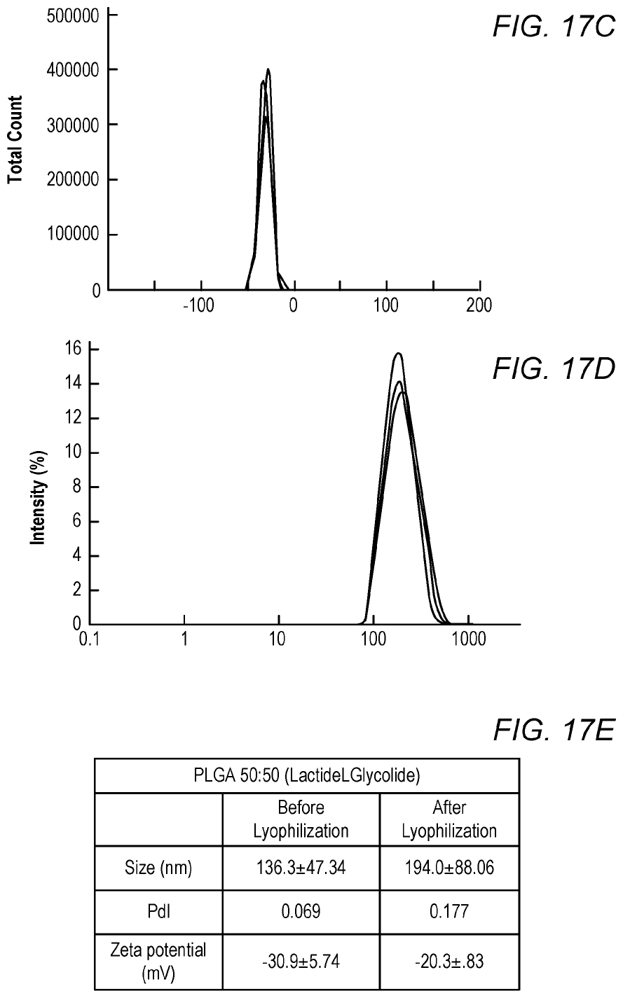

To develop solid particles for the solubilization and encapsulation of AD-3281 that can increase the solubility and cellular bioavailability, we formulated AD-3281 into PLGA nanoparticles using the emulsification evaporation method (FIG. 17). PLGA nanoparticles contained 0.1-0.3% (w/w) free AD-3281 as determined by high-performance liquid chromatography HPLC. (FIG. 17). Particle size were determined in dynamic light scattering DLS and the Z potential Zeta potential of the PLGA nanoparticles was measured with Zeta-sizer DLS (Malvern Instruments, UK). The morphology of the nanoparticles was determined using TEM, showing uniform spherical structures. For TEM analysis, samples were placed on glow discharged carbon coated 300 mesh copper TEM grids (Ted Pella, Inc.). After blotting, the samples were negatively stained with a 2% aqueous solution of uranyl acetate for 30 sec and air-dried. The samples were examined by FEI Tecnai 12 G2TWIN TEM operated at 120 kV.

### Nanoparticles Uptake

We evaluated the uptake of the polymeric nanoparticles by A375. A375 were incubated with PLGA nanoparticles which is encapsulated with 6-coumarin for 0 min, 4, 7, and 24 h. Cells were washed, resuspended in 0.2 ml FACS buffer (PBS with 1% FCS and 0-1% sodium azide), and analyzed using Flow cytometry (Beckman Coulter). Analysis was done using FCS Express 5 Flow research edition (De Novo software). This assay confirmed a maximal uptake after 7 h of incubation by using fluorescence-activated cell analyzer (FIG. 17).

### Anti-Proliferative Activity of AD-3281 Encapsulated Nanoparticles

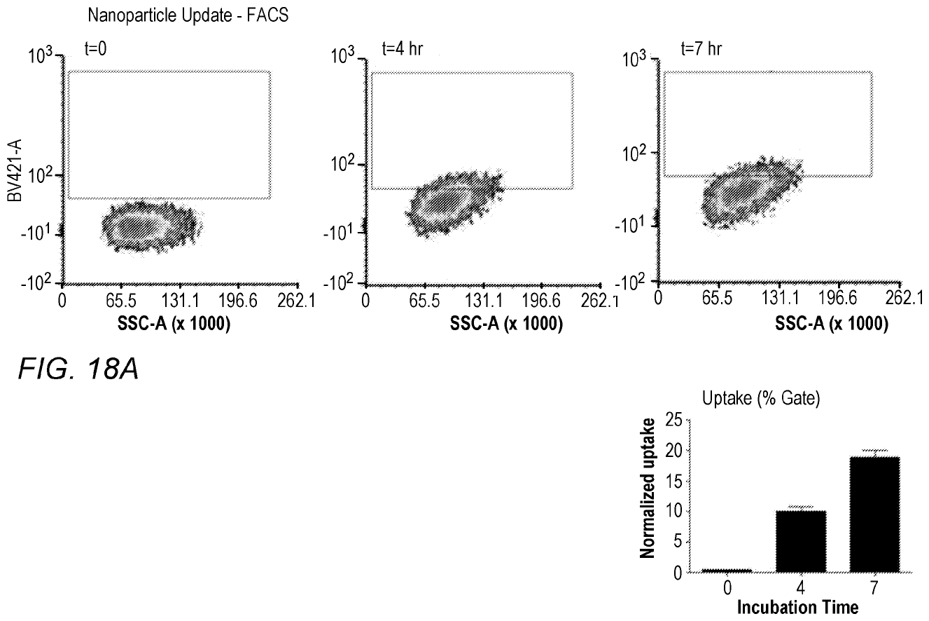

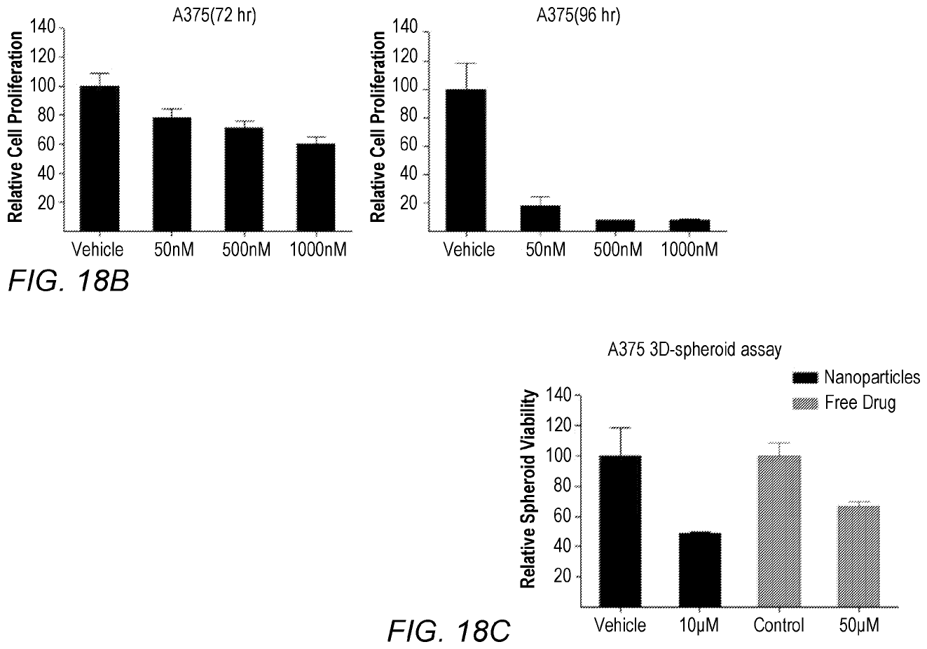

We assessed the effect of the encapsulated AD-3281 on the proliferation of endothelial cells. HUVECs were treated q.o.d with PLGA-AD-3281 (500-1000 nM AD-3281 equivalent) and the growth of HUVECs was evaluated using MTT assay after 5 days. Treated cells showed ˜31% inhibition in cell proliferation compared with cells exposed to vehicle only. Similarly, the effect of AD-3281 nano-formulation on the proliferation of A375 cancer cells was tested. After 72 h a dose-dependent inhibition was observed in treated cells with the encapsulated AD-3281 (500 mM-1000 mM AD-3281 equivalent) (22-40%). The incubation period of 96 h showed an effective reduction in the growth of A375 cells by 82-92%. (FIG. 18b). The interaction of the nanoparticles with cells were investigated using uptake assays confirming the uptake of polymeric nanoparticles by A375 with a level of ˜20% after 7 hours of incubation (FIG. 18b).

### AD-3281 Suppresses the Growth of A375 Spheroids

The inhibition activity of AD-3281 on A375 cells was evaluated in 3D multicellular spheroids that exhibit spatial cell-cell interaction, proliferation, and show gradient of nutrients due to diffusion of drugs similarly to in vivo. Therefore, 3D spheroid are expected to be a better predictive to the performance in vivo. Spheroid viability was measured using WST1 assay; spheroids were exposed to 10 μM equivalent to AD-3281 and to and 50 μM free inhibitor. After 96 h, spheroids treated with encapsulated inhibitor induced a reduction of 51% in spheroids viability compared to spheroids treated with vehicle only. Spheroids treated with the free inhibitor did not show a significant reduction of the spheroid's viability. (FIG. 18b).

### Discussion

Tumor growth and metastasis depend on the angiogenesis process. The inhibition of angiogenesis was established as an important modality for tumor suppression and spread when combined with chemotherapeutic drugs. While there are range of inhibitors that reached clinical approval, many of them are not sufficiently efficient or carry various side effects. Therefore, finding new angiogenic inhibitors, with high potency and drug-like properties may open up new avenue in cancer treatment, especially given the lower toxicity profile of these agents compared with chemotherapies.

Many studies established that MetAP2 plays an important role in the development of various types of cancer and the specific downregulation of human MetAP2 expression by an antisense oligonucleotide were predominantly affect endothelial cell proliferation. In our recently paper, we found the involvement of MetAP2 in lymphangiogenesis, indicating a dual action of MetAp2 in both vascular and lymphatic capillary formation. Therefore, there is a rationale for positioning MetAp2 as a useful target for the treatment of primary cancers as well as metastatic disease.

One of the most effective known inhibitors of MetAp2 is originated from the natural compound Fumagillin. This small molecule was isolated from Aspergillus fumigatus Fresenius, and the synthetic analogue, O-(chloroacetylcarbamoyl) fumagillol or TNP-470, (also referred to as AGM-1470) was one of the most potent analogs of fumagillin as demonstrated in angiogenesis cell models and one of the first anti-angiogenic small molecule drugs to undergo clinical trials. However, the development of this derivate was hindered by major clinical drawbacks related to dose-depending side effects. The high potential of fumagillol derivates motivated us and others to search for new safe compounds with high activity.

We synthesized a series of derivatives based on the structure of fumagillin. The selected lead compound, AD-3281 was the most active in suppressing endothelial cell proliferation and arrest MeAp2 enzymatic activity. It is well established that MetAp2 is overexpressed in the tumor microenvironment, and most significantly in the endothelium. We found that MetAp2 was also highly expressed in cancer cells in comparable level to endothelial cells and in the enzymatic assay AD-3281 markedly suppressed the proteolytic activity of MeAp2 (FIG. 10). It was indicated that the inhibition of MetAp2 enzymatic activity leads to angiogenesis blockage and suppress tumor growth.

Furthermore, we aimed to investigate the effect of AD-3281 on cellular functionality in cancer and endothelial cells. MetAp2 inhibition in vascular endothelium is known to regulate cell proliferation through cell cycle arrest in the late G₁ phase. We found that the presence of AD-3281 in human melanoma cell line, A375, as well as the primary endothelial cells, HUVEC, impaired cell proliferation in range of concentration of 1-100 μM and endothelial tube formation in 100 μM. The in-vitro observations correlate with our in vivo results, AD-3281 showed substantial anti-tumor effects and it was observed in two tumor-bearing mice models. This shows the broad biological effect of AD-3281. Histological analyses showed that in mice treated with AD-3281 endothelial cells re-modulation was affected. Immunofluorescence of extracted murine tumors tissue sections showed that in treated tissues blood vessels positive cells were organized more sporadically and less collectively as vessels, compared with untreated groups. Moreover, AD-3281 reduced the cellular proliferation in treated tumors; this was detected by the nuclear marker ki-67.

Given the promising in vivo results, AD-3281 could be a great candidate for further development towards clinical studies. However, desired clinically translatable formulation would be such that maximize AD-3281 solubilization while utilizing minimal contents of organic solvents that might lead to adverse effects. Therefore, we developed a nanoparticle-based encapsulation of the active compound using the well-established emulsification technique to produce biodegradable PLGA nanoparticles loaded with AD-3281 with a mean size of ˜200 nm that are typically used for injectable particles for cancer therapy (FIG. 17). Our previous studies showed the capacity to enhance bioavailability of lipophilic drugs, as well as improving their retention and stability in vivo, using 10-100 folds less of a dose than the free drug. We aimed to explore the cellular availability in monolayer cultures and in 3D multicellular ones. In many cases shifting from free form of molecules to particulate carriers can trigger cell uptake via endocytosis, thus improving accessibility of the drugs to the cells. In agreement, we found that the relatively high concentrations that were required for suppressing cell proliferation in vitro was attributed to the limited transport into cells. The inhibition effect of the encapsulated AD-3281 on the proliferation of endothelial and A375 cells showed a significant improvement of bioavailability of AD-3281 with almost 20-fold reduction in dose compared with the free molecule (FIG. 18). Unlike 2D cell cultures, in 3D cell assemble and interact spatially thus showing more physiologically relevance and provide better prediction of drug efficacy compared to monolayer cell cultures. To generate a robust spheroid growth, we use a lab-designed 3D-printed multi-well array that can generate uniform 3D cultures with high reproducibility. In the spheroid model, encapsulated AD-3281 was found to significantly suppress spheroid growth, while the free AD-3281 showed a slight reduction of spheroids viability. This can be explained by the improvement of the penetration of AD-3281 in their particulate PLGA nano carrier form, that enables high capacity of trans and intra cellular transport.

Taken together, our data shows that our new inhibitor AD-3281 has promising therapeutic properties in the treatment of cancer progression; this novel inhibitor demonstrated an effective inhibition role in blood vascularization and tumor progression in tumor-bearing mice. These significant results were mainly attributed to its anti-angiogenic and anti-cancer activity. Our outcomes using AD-3281 emphasize it as a great potential compound for treating highly vascularized tumors it may be useful for cancer patients as a long-term maintenance drug to prevent tumor recurrence.

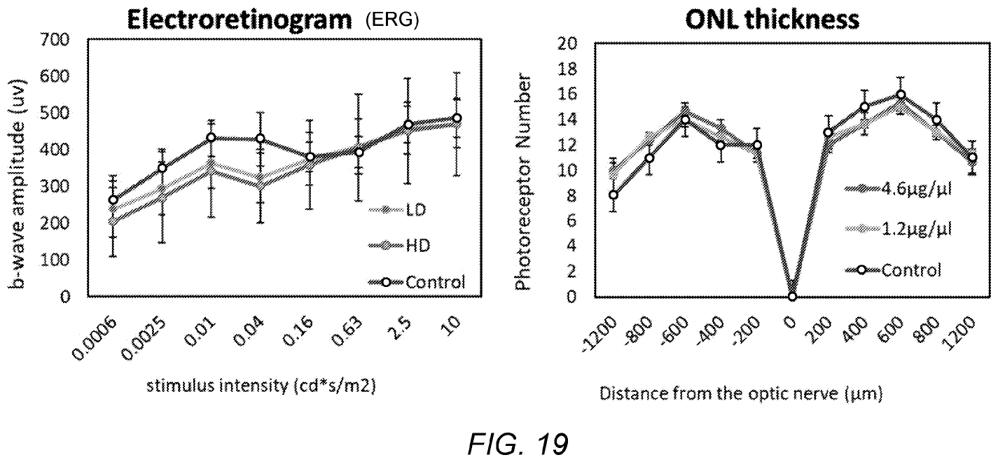

ERG study in mice revealed high safety of injected compound up to 4.6 μg/ul which is almost ×4 from the active dose in CNV model (FIG. 19).

1.-76. (canceled)

77. A compound of the general formula (I): 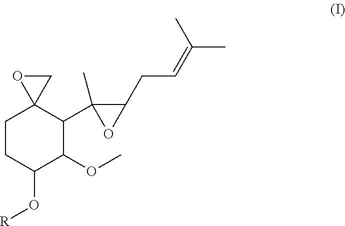
wherein
R is a carbocyclyl comprising a ring structure of two or more rings, selected from wherein the carbocyclyl group is an adamantly or a derivative thereof; a norbornanyl or a derivative thereof; a norbornenyl or a derivative thereof; a norbornenyl or a derivative thereof; a steroidyl or a derivative thereof; a camphoryl or a camphor derivative; a camphenyl or a camphene derivative; a tricyclo(2.2.1.0(2,6))heptanyl or a derivative thereof; or a tetracyclo[3.2.0.0(2,7).0(4,6)]heptanyl or a derivative thereof.

78. The compound according to claim 77, wherein variant R is bonded to the oxygen atom of the compound of Formula (I) directly or via a spacer or linker moiety X.

79. The compound according to claim 78, wherein the compound is of Formula (IA): 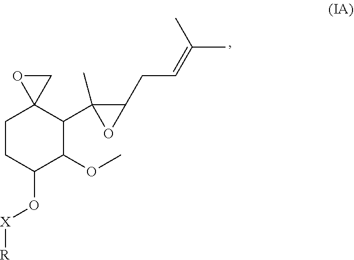
wherein R is the carbocyclyl and X is a functional group bridging carbocyclyl R and the oxygen atom of the structure of Formula (I).

80. The compound according to claim 79, wherein X is absent and R is directly bonded to the oxygen atom.

81. The compound according to claim 77, wherein a compound of structure (I) or (IA) is a compound of structure (IB): 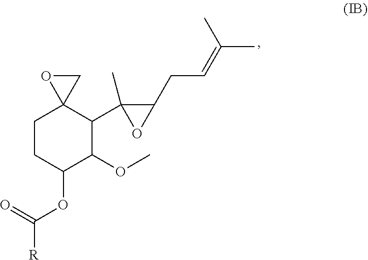
wherein R is the carbocycle.

82. The compound according to claim 77, wherein the compound of Formula (I) or (IA) is a compound designated compound (IC): 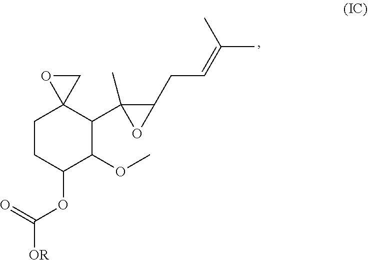
wherein R is the carbocycle.

83. The compound according to claim 77, wherein the compound of Formula (I) or (IA) is a compound of Formula (ID): 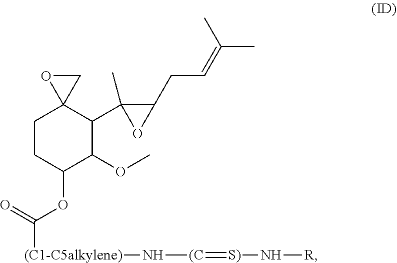
wherein R is the carbocyclyl.

84. The compound according to claim 77, wherein a compound of Formula (I) or (IA) is a compound of Formula (IE): 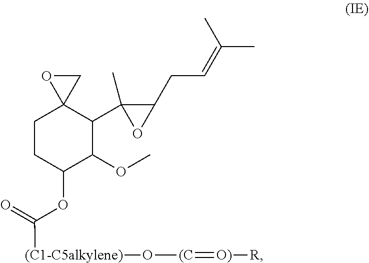
wherein R is the carbocyclyl.

85. The compound according to claim 77, wherein a compound of Formula (I) or (IA) is a compound of Formula (IF): 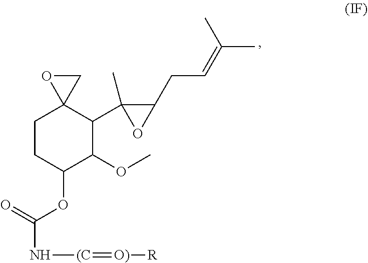
wherein R is the carbocyclyl.

86. The compound according to claim 77, wherein a compound of Formula (I) or (IA) is a compound of Formula (IG): 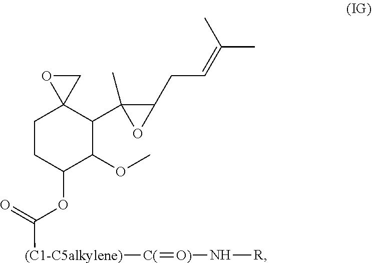
wherein R is the carbocyclyl.

87. The compound according to claim 77, wherein a compound of Formula (I) or (IA) is a compound of Formula (IH): 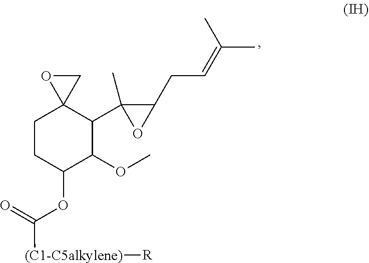
wherein R is the carbocycle.

88. The compound according to claim 77, wherein R is adamantly of structure (A1) or (A2): 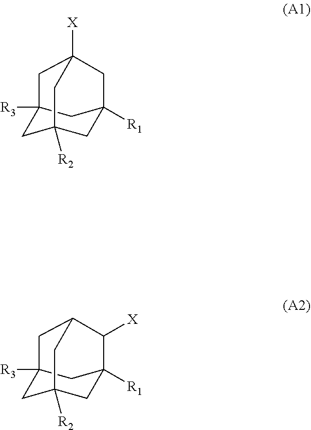
wherein for each of structure (A1) and (A2), independently:
X is a functional group or an atom associating the adamantly group with the oxygen atom of the compound of Formula (I); and wherein
each of R₁, R₂ and R₃, independently of the other, is —H, or is selected from halide (F, Cl, Br, I), —C₁-C₃alkyl, —C₂-C₄alkenyl, —C₁-C₃alkylhalide, hydroxyl, carboxyl, carboxylate, —C₆-C₁₀aryl, hydroxyalkyl, sulfate, sulfonate, sulfonamide, and sulfonic acid.

89. The compound according to claim 88, wherein the 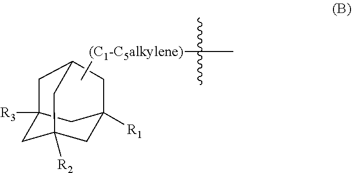
wherein
each of R₁, R₂ and R₃, independently of the other, is H, or is selected from —C₁-C₃alkyl, —C₂-C₄alkenyl, —C₁-C₃alkylhalide, hydroxyl, carboxyl, carboxylate, —C₆-C₁₀aryl, hydroxyalkyl and halide; and [symbol]
indicates a point of connectivity to the oxygen atom of a structure of Formula (I).

90. The compound according to claim 88, wherein the 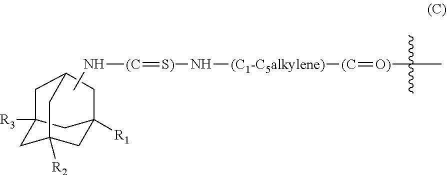
wherein
each of R₁, R₂ and R₃, independently of the other, is H, or is selected from —C₁-C₃alkyl, —C₂-C₄alkenyl, —C₁-C₃alkylhalide, hydroxyl, carboxyl, carboxylate, —C₆-C₁₀aryl, hydroxyalkyl and halide; and [symbol]
indicates a point of connectivity to the oxygen atom of a structure of Formula (I).

91. The compound according to claim 88, wherein the 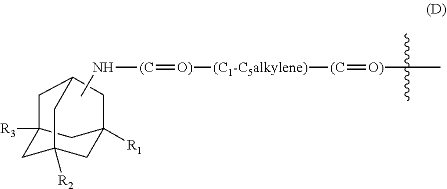
wherein
each of R₁, R₂ and R₃, independently of the other, is H, or is selected from —C₁-C₃alkyl, —C₂-C₄alkenyl, —C₁-C₃alkylhalide, hydroxyl, carboxyl, carboxylate, —C₆-C₁₀aryl, hydroxyalkyl and halide; and [symbol]
indicates a point of connectivity to the oxygen atom of a structure of Formula (I).

92. The compound according to claim 77, wherein a compound of Formula (I) is selected from compounds herein designated:
AD-3302;
AD-3281;
AD-3306;
AD-3283;
AD-3305;
AD-3301;
AD-3295;
AD-3294;
AD-3286;
AD-3287; and
AD-3290.

93. The compound according to claim 77, wherein the carbocyclyl is of structure (E): 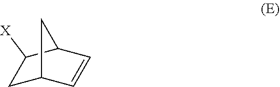
or the carbocyclyl is of structure (F): 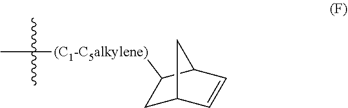
wherein the —C₁-C₅alkylene is selected from methylene, ethylene, propylene, butylene and pentylene, or the carbocyclyl is of structure (G): 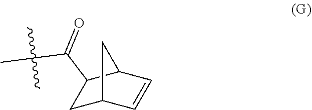 [SMILES: *C(=O)C1CC2C=CC1C2]
wherein the dashed line designates the bond of connectivity to the oxygen atom.

94. A composition comprising a compound according to claim 77.

95. A pharmaceutical composition comprising one or more compound according to claim 77, for administration by oral, buccal, sublingual, rectal, nasal, topical, transdermal, vaginal, parenteral, subcutaneous, intramuscular, intravenous or intradermal administration.

96. A method for reducing or diminishing or preventing a biological effect associated with activity of MetAp2 in a subject, the method comprising administering to said subject an effective amount of a compound of claim 77; or
a method of preventing or treating angiogenesis, an angiogenesis-related disease or an angiogenesis-dependent disease in a subject, the method comprising administering to the subject an effective amount of a compound according to claim 77; or
a method for preventing or treating a cancer in a subject, the method comprising administering to the subject an effective amount of a compound according to claim 77; or
a method for preventing or treating a pulmonary and hepatic fibrosis in a subject, the method comprising administering to the subject an effective amount of a compound according to claim 77; or
a method for preventing or treating a disease in a subject, the method comprising administering to the subject an effective amount of a compound according to claim 77, wherein the disease is selected from angiogenesis, ocular angiogenesis, ocular neovascular diseases, wounds, chronic ulcer, ischemic stroke, myocardial infarction, angina pectoris, peripheral artery disease, critical limb ischemia, diabetic foot ulcer, cerebrovascular dementia, cancer, pulmonary fibrosis, hepatic fibrosis, endometriosis, arthritis, autoimmune diseases, obesity and microsporidiosis; or
a method of preventing or treating at least one ocular or dermal disease or condition in a subject, the disease or condition being associated with MetAp2 activity, the method comprising ocular or dermal delivery of a composition comprising at least one MetAp2 inhibitor compound according to claim 77.
